SEQUENCE LISTING (23 sequences)

SEQ ID NO: 1 - DNA, 301 bp, Human
ggaaccaaggcaggatgaagacattgcctgtgctcgtgctgtctcttaccttactgactgtcttctcagagacctccccgattttgacagagaagcaggccaaacagctcctaagatctcggcgacaggatagaccaagcaaacccggattccccgatgagccaatgcgggaatacatgcaccacctgctcgccctggagcatcgcgctgaggagcagttcctggagcactggctgaaccctcactgcaaaccccactgtgacaggaacaggattcatcctgtgtaagggggatcaggacc

SEQ ID NO: 2 - protein, 90 aa, Human
MKTLPVLVLSLTLLTVFSETSPILTEKQAKQLLRSRRQDRPSKPGFPDEPMREYMHHLLALEHRAEEQFLEHWLNPHCKPHCDRNRIHPV

SEQ ID NO: 3 - DNA, 270 bp, Human
atgaagacattgcctgtgctcgtgctgtctcttaccttactgactgtcttctcagagacctccccgattttgacagagaagcaggccaaacagctcctaagatctcggcgacaggatagaccaagcaaacccggattccccgatgagccaatgcgggaatacatgcaccacctgctcgccctggagcatcgcgctgaggagcagttcctggagcactggctgaaccctcactgcaaaccccactgtgacaggaacaggattcatcctgtg

SEQ ID NO: 4 - protein, 70 aa, Human
SPILTEKQAKQLLRSRRQDRPSKPGFPDEPMREYMHHLLALEHRAEEQFLEHWLNPHCKPHCDRNRIHPV

SEQ ID NO: 5 - DNA, 210 bp, Human
tccccgattttgacagagaagcaggccaaacagctcctaagatctcggcgacaggatagaccaagcaaacccggattccccgatgagccaatgcgggaatacatgcaccacctgctcgccctggagcatcgcgctgaggagcagttcctggagcactggctgaaccctcactgcaaaccccactgtgacaggaacaggattcatcctgtg

SEQ ID NO: 6 - protein, 53 aa, Human
QDRPSKPGFPDEPMREYMHHLLALEHRAEEQFLEHWLNPHCKPHCDRNRIHPV

SEQ ID NO: 7 - DNA, 159 bp, Human
caggatagaccaagcaaacccggattccccgatgagccaatgcgggaatacatgcaccacctgctcgccctggagcatcgcgctgaggagcagttcctggagcactggctgaaccctcactgcaaaccccactgtgacaggaacaggattcatcctgtg

SEQ ID NO: 8 - protein, 15 aa, Human
SPILTEKQAKQLLRS

SEQ ID NO: 9 - DNA, 45 bp, Human
tccccgattttgacagagaagcaggccaaacagctcctaagatct

SEQ ID NO: 10 - DNA, 22 bp, Artificial Sequence
  Primer
ggaaccaaggcaggatgaagac

SEQ ID NO: 11 - DNA, 22 bp, Artificial Sequence
  Primer
ggtcctgatcccccttacacag

SEQ ID NO: 12 - DNA, 47 bp, Artificial Sequence
  Primer
ctagaattccaccatgaagacattgcctgtgctcgtgctgtctctta

SEQ ID NO: 13 - DNA, 40 bp, Artificial Sequence
  Primer
gatccgtcgaccacaggatgaatcctgttcctgtcacagt

SEQ ID NO: 14 - protein, 99 aa, Artificial Sequence
  amino acid sequence of the polypeptide encoded by the expression vector constructed in Example 3
MKTLPVLVLSLTLLTVFSETSPILTEKQAKQLLRSRRQDRPSKPGFPDEPMREYMHHLLALEHRAEEQFLEHWLNPHCKPHCDRNRIHPVVDYKDDDDK

SEQ ID NO: 15 - DNA, 297 bp, Artificial Sequence
  DNA fragment encoding DRL90h-FLAG fused protein
atgaagacattgcctgtgctcgtgctgtctcttaccttactgactgtcttctcagagacctccccgattttgacagagaagcaggccaaacagctcctaagatctcggcgacaggatagaccaagcaaacccggattccccgatgagccaatgcgggaatacatgcaccacctgctcgccctggagcatcgcgctgaggagcagttcctggagcactggctgaaccctcactgcaaaccccactgtgacaggaacaggattcatcctgtggtcgactacaaggacgacgatgacaag

SEQ ID NO: 16 - DNA, 43 bp, Artificial Sequence
  Primer
tcctggtcgacttacacaggatgaatcctgttcctgtcacagt

SEQ ID NO: 17 - protein, 11 aa, Artificial Sequence
  Synthetic Peptide CPP1-1
QDRPSKPGFPC

SEQ ID NO: 18 - protein, 11 aa, Artificial Sequence
  Synthetic Peptide CPP1-2
CSPILTEKQAK

SEQ ID NO: 19 - DNA, 19 bp, Artificial Sequence
  Primer
ggtcctgatccccttacac

SEQ ID NO: 20 - DNA, 29 bp, Artificial Sequence
  Primer
aggagaattccaccatggagaagctgttt

SEQ ID NO: 21 - DNA, 28 bp, Artificial Sequence
  Primer
ccccagtcgactcacacaggatggacaa

SEQ ID NO: 22 - protein, 90 aa, Mouse
MEKLFVLVFALALLAFSSDASPILTEKQAKQLLRSRRQDRPNKPGFPDEPMREYMHHLLALEHRAEEQFLEHWLNPHCKPHCDRNIVHPV

SEQ ID NO: 23 - DNA, 270 bp, Mouse
atggagaagctgtttgtcttggtctttgctctcgccctgctggctttctcctcagatgcctccccgattctgacagaaaagcaggcaaaacagctcctgaggtccaggcgacaggacaggcccaacaaacctggattcccggacgagcccatgcgggagtatatgcatcatctcctggccctggagcaccgtgctgaggagcagtttctggagcactggctgaatcctcactgcaagccgcactgtgacaggaacattgtccatcctgtg

(19) United States
(12) Patent Application Publication
(10) Pub. No.: US 2004/0053276 A1
(43) Pub. Date: Mar. 18, 2004

(54) Novel tissue-specific secretory polypeptide and dna thereof

(76) Inventors: Yasuaki Ito, Ibaraki (JP); Hideyuki Tanaka, Ibaraki (JP); Atsushi Nishimura, Ibaraki (JP); Kazuhiro Ogi, Ibaraki (JP)

Correspondence Address: Mark Chao, Intellectual Department, Takeda Pharmaceutical North America Inc, Suite 500 475 Half Day Road, Lincolnshire, IL, 60069, US

(21) Appl. No.: 10/381,089

(22) PCT Filed: Sep. 21, 2001
(86) PCT No.: PCT/JP01/08223

(30) Foreign Application Priority Data
Sep. 22, 2000 (JP) 2000-293985
Sep. 29, 2000 (JP) 2000-302839

(51) Int. Cl. C12Q 1/68; C07H 21/04; C07K 14/47

(52) U.S. Cl. 435/6; 435/69.1; 435/320.1; 435/325; 530/350; 536/23.5

(57) ABSTRACT

The present invention aims at providing a novel, tissue-specific secretory polypeptide and a DNA encoding the same.

The polypeptide and the DNA encoding the same of the invention may be used for diagnosing, treating or preventing diseases, for example, such as cancers, immunological diseases, infections, gastrointestinal diseases, circulatory organ diseases and endocrine diseases. The polypeptide of the invention is also useful as a reagent for screening for compounds, or salts thereof, that promote or inhibit the activity of the polypeptide of the invention. Further, since an antibody to the polypeptide of the invention is able to recognize the polypeptide of the invention specifically, the antibody may be used for the detection, quantitative determination or neutralization of the polypeptide of the invention in a sample liquid.

### TECHNICAL FIELD

The present invention relates to a novel, secretory polypeptide regulating biological functions, and a DNA encoding the polypeptide; an antibody to the polypeptide; a method and a kit for screening for compounds that promote or inhibit the activity of the polypeptide; a compound obtainable by using the screening method or the screening kit; a medicine comprising the compound; use thereof; and a non-human transgenic animal harboring the DNA.

### BACKGROUND ART

Regardless of being prokaryotic or eukaryotic, cells secrete a wide variety of proteins by their inherent mechanisms. In particular, multi-cellular organisms (living bodies) exchange various information among cells in order to maintain their differentiation, proliferation and homeostasis; and various humoral factors that play major roles in such exchange of information are secretory proteins or their mature proteins for the most part. Such proteins are grouped into hormones, neurotransmitters, cytokines, growth factors and so forth according to their structural or functional characteristics (Cytokines/Growth Factors in “Library for BIO Science Terms”, Yohdo-sha). With advances in the recombinant DNA technology and the cell culturing technology, genes encoding these secretory proteins and structures of such proteins have been steadily elucidated. On the other hand, the discovery of such factors brings about a rapid progress in the analysis of their receptors expressed on cell surfaces, leading to elucidation of mechanisms of signal transfer in individual cells and characterization of the physiological functions of such receptors (“Receptors on Cell Membrane: from Basic Knowledge to Latest Information”, Nanzando). In a great number of human diseases and disease states of various disease model animals, it is found frequently that abnormal expression of some humoral factors, which should naturally retain homeostasis, is the cause of the disease or disease state, or leads to worsening of the disease or disease state. Besides, there are phenomena that may be applicable to the field of diagnosis of various diseases, e.g., the so-called tumor markers whose expression is enhanced specifically in cancers (“Tumor Markers”, Chugai-Igaku Co.). Mechanisms of their expression control have become important targets in researches toward drug development.

Currently, the analysis of total DNA possessed by one organism, i.e. genomic DNA is being performed in a number of organisms ranging from prokaryotes to eukaryotes. The analysis has already been completed in Escherichia coli, yeast, Caenorhabditis elegans, Drosophila and human. As a result of this analysis, it has been found that human genome contains 30,000 to 40,000 genes (Nature, Vol. 409, No. 6822 (2001); Science, Vol. 291, No. 5507 (2001)).

Although a great number of genes encoding secretory proteins or secretory peptides have been isolated, it is hard to say that they cover all such proteins or peptides in view of the total genome. For understanding a life phenomenon of the level of an individual, every information exchange between cells in the individual must be explainable. It is very likely that, in addition to those known gene, unknown humoral functional molecules are playing important physiological roles, and discovery of such substances has been strongly desired.

It is an object of the present invention to provide a novel secretory polypeptide regulating biological functions, or an amide, ester or salt thereof (hereinafter, sometimes referred to as “DRL90 polypeptide” or just “DRL90”, or sometimes simply referred to as “the polypeptide of the invention”); a mature form of the polypeptide or an amide, ester or salt thereof (hereinafter, sometimes simply referred to as “the peptide of the invention”); a partial peptide of the above polypeptide or peptide, or an amide, ester or salt of the partial peptide (hereinafter, sometimes simply referred to as “the partial peptide of the invention”); a polynucleotide or DNA encoding the above polypeptide, peptide or partial peptide (hereinafter, sometimes referred to simply as “the DNA of the invention”); a recombinant vector; a transformant; a method of producing the above polypeptide, peptide or partial peptide; a medicine comprising the above polypeptide, peptide or partial peptide or the above polynucleotide; an antibody to the above polypeptide, peptide or partial peptide; and a screening method using the above polypeptide, peptide or partial peptide.

### DISCLOSURE OF THE INVENTION

The isolation of a novel, secretory protein regulating biological functions will give new findings on those mechanisms involved in cell differentiation, cell proliferation, biological defense, canceration, etc. and thus will be able to promote elucidation of life phenomena such as ontogeny and the maintenance of homeostasis. Also, development of new medicines that would exhibit inhibitory or promotional effect on the protein and would be useful for the prevention, diagnosis or treatment of various diseases can be expected.

As a result of extensive and intensive researches, the present inventors have succeeded in cloning a cDNA having a novel nucleotide sequence from a human thymus cDNA library. The inventors have found that the protein encoded by the obtained cDNA is a secretory humoral factor produced in those organs important for the maintenance of homeostasis, biological defense and reproduction, such as thymus, spleen, liver, pancreas, lymph nodes, lymphocytes, leukocytes and testis, and that this protein is useful as an agent for regulating the differentiation and/or function of germ cells. As a result of further investigation based on these findings, the present invention has been achieved.

The present invention provides:

(1) A polypeptide comprising an amino acid sequence identical or substantially identical with the amino acid sequence as shown in SEQ ID NO: 4, or an amide, ester or salt thereof.

(2) The polypeptide according to (1) comprising the amino acid sequence as shown in SEQ ID NO: 4, SEQ ID NO: 2 or SEQ ID NO: 14, or an amide, ester or salt thereof.

(3) A peptide comprising an amino acid sequence identical or substantially identical with the amino acid sequence as shown in SEQ ID NO: 6 or SEQ ID NO: 8, or an amide, ester or salt thereof.

(4) The peptide according to (3) comprising the amino acid sequence as shown in SEQ ID NO: 6 or SEQ ID NO: 8, or an amide, ester or salt thereof.

(5) A partial peptide of the polypeptide according to (1) or the polypeptide according to (3), or an amide, ester or salt of the partial peptide.

(6) A polynucleotide comprising a polynucleotide having a nucleotide sequence encoding the polypeptide according to (1).

(7) The polynucleotide according to (6) comprising a polynucleotide having the nucleotide sequence as shown in SEQ ID NO: 1, SEQ ID NO: 3, SEQ ID NO: 5 or SEQ ID NO: 15.

(8) The polynucleotide according to (6) or (7), wherein the polynucleotide is DNA.

(9) A polynucleotide comprising a polynucleotide having a nucleotide sequence encoding the peptide according to (3).

(10) The polynucleotide according to (9) comprising a nucleotide having the nucleotide sequence as shown in SEQ ID NO: 7 or SEQ ID NO: 9.

(11) The polynucleotide according to (9) or (10), wherein the polynucleotide is DNA.

(12) A recombinant vector comprising the polynucleotide according to (6) or (9).

(13) A transformant transformed with the recombinant vector according to (12). (14) A method for producing the polypeptide according to (1) or an amide, ester or salt thereof or the peptide according to (3) or an amide, ester or salt thereof, comprising culturing the transformant and allowing the polypeptide according to (1) or the peptide according to (3) to be produced and accumulated.

(15) An antibody to the polypeptide according to (1) or an amide, ester or salt thereof, the peptide according to (3) or an amide, ester or salt thereof, or the partial peptide according to (5) or an amide, ester or salt thereof.

(16) A method for screening for compounds, or salts thereof, that promote or inhibit the activity of the polypeptide according to (1) or an amide, ester or salt thereof, the peptide according to (3) or an amide, ester or salt thereof, or the partial peptide according to (5) or an amide, ester or salt thereof, wherein the method is characterized by using the polypeptide according to (1) or an amide, ester or salt thereof, the peptide according to (3) or an amide, ester or salt thereof, or the partial peptide according to (5) or an amide, ester or salt thereof.

(17) A kit for screening for compounds, or salts thereof, that promote or inhibit the activity of the polypeptide according to (1) or an amide, ester or salt thereof, the peptide according to (3) or an amide, ester or salt thereof, or the partial peptide according to (5) or an amide, ester or salt thereof, which comprises the polypeptide according to (1) or an amide, ester or salt thereof, the peptide according to (3) or an amide, ester or salt thereof, or the partial peptide according to (5) or an amide, ester or salt thereof.

(18) A compound, or a salt thereof, that promotes or inhibits the activity of the polypeptide according to (1) or an amide, ester or salt thereof, the peptide according to (3) or an amide, ester or salt thereof, or the partial peptide according to (5) or an amide, ester or salt thereof, which is obtainable by using the screening method according to (16) or the screening kit according to (17).

(19) A medicine comprising the compound according to (18) or a salt thereof.

(20) A prophylactic and/or therapeutic agent for cancers, immunological diseases, infections, digestive tract diseases, circulatory organ diseases or endocrine diseases, comprising a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(21) A prophylactic and/or therapeutic agent for sterility, comprising a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(22) An agent for regulating the differentiation and/or function of germ cells, comprising a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(23) An agent for regulating the differentiation and/or proliferation of blood cells, comprising a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(24) A medicine comprising the polypeptide according to (1) or an amide, ester or salt thereof, or the peptide according to (3) or an amide, ester or salt thereof.

(25) The medicine according to (24), wherein the medicine is a prophylactic and/or therapeutic agent for cancers, immunological diseases, infections, digestive tract diseases, circulatory organ diseases or endocrine diseases.

(26) The medicine according to (24), wherein the medicine is a prophylactic and/or therapeutic agent for sterility.

(27) The medicine according to (24), wherein the medicine is an agent for regulating the differentiation and/or function of germ cells.

(28) The medicine according to (24), wherein the medicine is an agent for regulating the differentiation and/or proliferation of blood cells.

(29) A medicine comprising the polynucleotide according to (6) or (9).

(30) The medicine according to (29), wherein the medicine is a prophylactic and/or therapeutic agent for cancers, immunological diseases, infections, digestive tract diseases, circulatory organ diseases or endocrine diseases.

(31) The medicine according to (29), wherein the medicine is a prophylactic and/or therapeutic agent for sterility.

(32) The medicine according to (29), wherein the medicine is an agent for regulating the differentiation and/or function of germ cells.

(33) The medicine according to (29), wherein the medicine is an agent for regulating the differentiation and/or proliferation of blood cells.

(34) A diagnostic agent comprising the antibody according to (15).

(35) A medicine comprising the antibody according to (15).

(36) An antisense DNA having a nucleotide sequence, or a part thereof, complementary or substantially complementary to the DNA encoding the polypeptide according to (1) or the peptide according to (3), the antisense DNA having an effect capable of inhibiting the expression of the DNA.

(37) A medicine comprising the antisense DNA according to (36).

(38) A dimer of a polypeptide comprising an amino acid sequence identical or substantially identical with the amino acid sequence as shown in SEQ ID NO: 4; or an amide, ester or salt of the dimer.

(39) A dimer composed of two polypeptides comprising the amino acid sequence as shown in SEQ ID NO: 4, wherein the respective C-terminal cysteine residues of the two polypeptides are linked to each other; or an amide, ester or salt of the dimer.

(40) A dimer of a peptide comprising an amino acid sequence identical or substantially identical with the amino acid sequence as shown in SEQ ID NO: 6; or an amide, ester or salt of the dimer.

(41) A dimer composed of two peptides comprising the amino acid sequence as shown in SEQ ID NO: 6, wherein the respective C-terminal cysteine residues of the two peptides are linked to each other; or an amide, ester or salt of the dimer.

(42) A dimer composed of a polypeptide comprising an amino acid sequence identical or substantially identical with the amino acid sequence as shown in SEQ ID NO: 4 and a peptide comprising an amino acid sequence identical or substantially identical with the amino acid sequence as shown in SEQ ID NO: 6; or an amide, ester or salt of the dimer.

(43) A dimer composed of a polypeptide comprising the amino acid sequence as shown in SEQ ID NO: 4 and a peptide comprising the amino acid sequence as shown in SEQ ID NO: 6; or an amide, ester or salt of the dimer.

(44) A method of preventing and/or treating cancers, immunological diseases, infections, digestive tract diseases, circulatory organ diseases or endocrine diseases, which is characterized by administering to a mammal an effective amount of the polypeptide according to (1) or an amide, ester or salt thereof or the peptide according to (3) or an amide, ester or salt thereof.

(45) A method of preventing and/or treating sterility, which is characterized by administering to a mammal an effective amount of the polypeptide according to (1) or an amide, ester or salt thereof or the peptide according to (3) or an amide, ester or salt thereof.

(46) A method of regulating the differentiation and/or function of germ cells, which is characterized by administering to a mammal an effective amount of the polypeptide according to (1) or an amide, ester or salt thereof or the peptide according to (3) or an amide, ester or salt thereof.

(47) A method of regulating the differentiation and/or proliferation of blood cells, which is characterized by administering to a mammal an effective amount of the polypeptide according to (1) or an amide, ester or salt thereof or the peptide according to (3) or an amide, ester or salt thereof.

(48) A method of preventing and/or treating cancers, immunological diseases, infections, digestive tract diseases, circulatory organ diseases or endocrine diseases, which is characterized by administering to a mammal an effective amount of the polynucleotide according to (6) or (9).

(49) A method of preventing and/or treating sterility, which is characterized by administering to a mammal an effective amount of the polynucleotide according to (6) or (9).

(50) A method of regulating the differentiation and/or function of germ cells, which is characterized by administering to a mammal an effective amount of the polynucleotide according to (6) or (9).

(51) A method of regulating the differentiation and/or proliferation of blood cells of germ cells, which is characterized by administering to a mammal an effective amount of the polynucleotide according to (6) or (9).

(52) A method of preventing and/or treating cancers, immunological diseases, infections, digestive tract diseases, circulatory organ diseases or endocrine diseases, which is characterized by administering to a mammal an effective amount of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(53) A method of preventing and/or treating sterility, which is characterized by administering to a mammal an effective amount of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(54) A method of regulating the differentiation and/or function of germ cells, which is characterized by administering to a mammal an effective amount of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(55) A method of regulating the differentiation and/or proliferation of blood cells, which is characterized by administering to a mammal an effective amount of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17).

(56) Use of the polypeptide according to (1) or an amide, ester or salt thereof or the peptide according to (3) or an amide, ester or salt thereof for producing a prophylactic and/or therapeutic agent for cancers, immunological diseases, infections, digestive tract diseases, circulatory organ diseases or endocrine diseases.

(57) Use of the polynucleotide according to (6) or (9) for producing a prophylactic and/or therapeutic agent for cancers, immunological diseases, infections, digestive tract diseases, circulatory organ diseases or endocrine diseases.

(58) Use of the polynucleotide according to (6) or (9) for producing a prophylactic and/or therapeutic agent for sterility.

(59) Use of the polynucleotide according to (6) or (9) for producing an agent for regulating the differentiation and/or function of germ cells.

(60) Use of the polynucleotide according to (6) or (9) for producing an agent for regulating the differentiation and/or proliferation of blood cells.

(61) Use of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17), for producing a prophylactic and/or therapeutic agent for cancers, immunological diseases, infections, digestive tract diseases, circulatory organ diseases or endocrine diseases.

(62) Use of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17), for producing a prophylactic and/or therapeutic agent for sterility.

(63) Use of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17), for producing an agent for regulating the differentiation and/or function of germ cells.

(64) Use of a compound, or a salt thereof, that promotes the activity of the polypeptide according to (1) or an amide, ester or salt thereof, the peptide according to (3) or an amide, ester or salt thereof, or the partial peptide according to (5) or an amide, ester or salt thereof, the compound or salt thereof being obtainable by using the screening method according to (16) or the screening kit according to (17), for producing an agent for regulating the differentiation and proliferation of blood cells.

(65) A non-human transgenic animal harboring the DNA according to (7) or (10) which is a foreign DNA or a mutant DNA thereof.

(66) The animal according to (65), wherein the non-human animal is a rodent.

(67) The animal according to (66), wherein the rodent is mouse or rat.

(68) A recombinant vector comprising the DNA according to (7) or (10) which is a foreign DNA or a mutant DNA thereof, wherein the vector is capable of expression in a non-human animal.

Further, the present invention provides:

(69) The polypeptide according to (1) or an amide, ester or salt thereof, wherein the amino acid sequence substantially identical with the amino acid sequence as shown in SEQ ID NO: 4 is an amino acid sequence having about 50% or more (preferably about 60% or more, more preferably about 70% or more, still more preferably about 80% or more, especially preferably about 90% or more, and most preferably about 95% or more) homology to the amino acid sequence as shown in SEQ ID NO: 4, and (70) The polypeptide according to (1) or an amide, ester or salt thereof, wherein the amino acid sequence substantially identical with the amino acid sequence as shown in SEQ ID NO: 4 is (i) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are deleted therefrom; (ii) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are added thereto; (iii) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are replaced with other amino acids; or (iv) an amino acid sequence which is a combination of these sequences.

Further, the polypeptide, the peptide, the partial peptide, the DNA and so forth of the invention may be applicable to molecular markers, tissue markers, chromosome mapping, identification of genetic diseases, diagnosis of disease states, or basic researches such as designing of primers or probes.

### BRIEF DESCRIPTION OF THE DRAWINGS

FIG. 1 shows the results of Northern blotting carried out to analyze tissues expressing the protein. “PBL” appearing in this Figure represents peripheral blood leukocytes.

FIG. 2 shows the results of dot blotting carried out to analyze tissues expressing the protein. In this Figure, the symbols have the following meanings: A1, brain; B1, cerebral cortex; C1, frontal lobe; D1, parietal lobe; E1, occipital lobe; F1, temporal lobe; G1, cerebral cortex; H1, pons; A2, left cerebellum; B2, right cerebellum; C2, corpus callosum; D2, amygdala; E2, caudate nucleus; F2, hippocampus; G2, medulla oblongata; H2, putamen; A3, nigra; B3, accumbens septi; C3, thalamus; D3, pituitary gland; E3, spinal cord; A4, heart; B4, aorta; C4, left atrium; D4, right atrium; E4, left ventricle; F4, right ventricle; G4, intraventricular septum; H4, the pointed end of heart; A5, esophagus; B5, stomach; C5, duodenum; D5, jejunum; E5, ileum; F5, ileocecum; G5, appendix; H5, ascending colon; A6, traverse colon; B6, descending colon; C6, rectum; A7, kidney; B7, skeletal muscle; C7, spleen; D7, thymus; E7, peripheral blood leukocytes; F7, lymph nodes; G7, bone marrow; H7, trachea; A8, lung; B8, placenta; C8, bladder; D8, uterus; E8, prostate; F8, testis; G8, ovary; A9, liver; B9, pancreas; C9, adrenal gland; D9, thyroid gland; E9, salivary gland; F9, mammary gland; A10, leukemia HL-60 cells; B10, HeLa cells; C10, leukemia K-562 cells; D10, leukemia MOLT-4 cells; E10, Burkitt's lymphoma Raji cells; F10, Burkitt's lymphoma Dauji cells; G10, adenocarcinoma AW480 cells; H10, lung cancer A549 cells; A11, fetal brain; B11, fetal heart: C11, fetal kidney; D11, fetal liver; E11, fetal spleen; F11, fetal thymus; G11, fetal lung; A12, yeast total RNA; B12, yeast total RNA; C12, E. coli ribosomal RNA; D12, E. coli DNA; G12, human DNA; and H12, human DNA.

FIG. 3 shows the results of Western blotting carried out for analyzing expression products.

FIG. 4 shows a schematic diagram for expression products.

### BEST MODE FOR CARRYING OUT THE INVENTION

A polypeptide comprising an amino acid sequence identical or substantially identical with the amino acid sequence as shown in SEQ ID NO: 4 (hereinafter, sometimes referred to as the “polypeptide of the invention”; sometimes, a polypeptide comprising an amino acid sequence identical or substantially identical with the amino acid sequence as shown in SEQ ID NO: 4 or an amide, ester or salt thereof may also be called the “polypeptide of the invention” collectively) may be a polypeptide derived from cells of any kind (e.g. hepatocytes, splenocytes, neurons, glia cells, pancreatic cells, bone marrow cells, mesangial cells, Langerhan's cells, epidermal cells, epithelial cells, endothelial cells, fibroblasts, fibrous cells, muscle cells, fat cells, immune cells (e.g. macrophages, T cells, B cells, natural killer cells, mast cells, neutrophils, basophils, eosinophils, monocytes), megakaryocytes, synovial cells, chondrocytes, osteocytes, osteoblasts, osteoclasts, mammary cells, interstitial cells, or progenitor cells of these cells, stem cells or cancer cells, etc.) of human or other warm-blooded animals (e.g. guinea pig, rat, mouse, chicken, rabbit, pig, sheep, bovine, monkey, etc.) or all tissues in which such cells are present, such as brain, various parts of brain (e.g. olfactory bulb, amygdaloid nucleus, cerebral basal nucleus, hippocampus, thalamus, hypothalamus, cerebral cortex, medulla oblongata, cerebellum), spinal cord, pituitary gland, stomach, pancreas, kidney, liver, gonad, thyroid, gall-bladder, bone marrow, adrenal gland, skin, muscle, lung, gastrointestinal tracts (e.g. large intestine, small intestine), blood vessels, heart, thymus, spleen, salivary gland, peripheral blood, prostate, testis, ovary, placenta, uterus, bone, cartilage, joint, skeletal muscle, etc. It may also be a recombinant polypeptide or a synthetic polypeptide.

When the polypeptide of the invention has a signal peptide (specifically, a polypeptide comprising an amino acid sequence identical or substantially identical with the amino acid sequence of SEQ ID NO: 4 to which a signal sequence consisting of 20 amino acids has been added to the N-terminal, as shown in SEQ ID NO: 2), the polypeptide may be efficiently secreted out of cells.

The term “substantially identical” means that the activity of the polypeptide (e.g. an activity (effect) of preventing and/or treating cancers, immunological diseases, infections, gastrointestinal diseases, circulatory organ diseases, endocrine diseases and fertility, an activity (effect) of regulating the differentiation and/or function of germ cells, or an activity (effect) of regulating the differentiation and/or proliferation of blood cells) or physiological characteristics of the polypeptide are substantially identical. Substitution, deletion, addition or insertion of amino acids often does not cause a significant change in physiological properties or chemical properties of a polypeptide. In such a case, the polypeptide that has undergone the substitution, deletion, addition or insertion may be said substantially identical with a corresponding polypeptide that has not undergone such substitution, deletion, addition or insertion. The substantially identical substituted amino acid in the above amino acid sequence may be selected from, for example, the other amino acids in the class to which the initial amino acid belongs.

Examples of non-polar (hydrophobic) amino acids include alanine, leucine, isoleucine, valine, proline, phenylalanine, tryptophan and methionine. Examples of polar (neutral) amino acids include glycine, serine, threonine, cysteine, tyrosine, asparagine and glutamine. Examples of positively charged (basic) amino acids include arginine, lysine and histidine. Examples of negatively charged (acidic) amino acids include aspartic acid and glutamic acid.

An amino acid sequence substantially identical with the amino acid sequence as shown in SEQ ID NO: 4 is not particularly limited as long as the polypeptide comprising the relevant amino acid sequence has an activity (nature) substantially identical with the activity of a polypeptide comprising the amino acid sequence as shown in SEQ ID NO: 4. For example, an amino acid sequence having about 50% or more, preferably about 60% or more, more preferably about 70% or more, still more preferably about 80% or more, especially preferably about 90% or more and most preferably about 95% or more homology to the amino acid sequence as shown in SEQ ID NO: 4 may be used. More specifically, the amino acid sequence as shown in SEQ ID NO: 2 or the amino acid sequence as shown in SEQ ID NO: 14 may be used, for example.

An example of the activity (nature) of substantially the same quality mentioned above may be that a polypeptide is secreted and acts as a humoral factor. The term “substantially the same quality” means that such a nature is qualitatively identical. Therefore, it is preferable that a nature such as secretory action or solubility is identical (e.g. about 0.1- to about 100-fold, preferably about 0.5- to about 10-fold, more preferably about 0.5- to about 2-fold), but quantitative elements such as the extent of that nature, the molecular weight of the polypeptide, etc. may be different.

Examples of the activity (nature) of substantially the same quality mentioned above include an activity that is qualitatively identical to the effect of preventing and/or treating cancers, immunological diseases, infections, gastrointestinal diseases, circulatory organ diseases, endocrine diseases and fertility; the effect of regulating the differentiation and/or function of germ cells; or the effect of regulating the differentiation and/or proliferation of blood cells possessed by the polypeptide comprising the amino acid sequence as shown in SEQ ID NO: 4.

More specific examples of polypeptides comprising the amino acid sequence as shown in SEQ ID NO: 4 include the so-called muteins, such as polypeptides comprising (i) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are deleted therefrom; (ii) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are added thereto; (iii) the amino acid sequence of SEQ ID NO: 4 wherein one or two or more amino acids (preferably about 1-30, more preferably about 1-20, still more preferably about 1-10, especially preferably about 1-5 and most preferably 1 or 2 amino acids) are replaced with other amino acids; or (iv) an amino acid sequence which is a combination of these sequences.

When the amino acid sequence is inserted, deleted or substituted as described above, the site of insertion, deletion or substitution is not particularly limited. However, positions other than the basic amino acid pairs and the cysteine residues in the amino acid sequence of SEQ ID NO: 4 may be preferable.

A peptide comprising an amino acid sequence identical or substantially identical with the amino acid sequence as shown in SEQ ID NO: 6 or SEQ ID NO: 8 (hereinafter, sometimes referred to as the “peptide of the invention”; sometimes, a peptide comprising an amino acid sequence identical or substantially identical with the amino acid sequence as shown in SEQ ID NO: 6 or SEQ ID NO: 8 or an amide, ester or salt of the peptide may also be called the “peptide of the invention” collectively) may be a peptide derived from cells of any kind (e.g. hepatocytes, splenocytes, neurons, glia cells, pancreatic cells, bone marrow cells, mesangial cells, Langerhan's cells, epidermal cells, epithelial cells, endothelial cells, fibroblasts, fibrous cells, muscle cells, fat cells, immune cells (e.g. macrophages, T cells, B cells, natural killer cells, mast cells, neutrophils, basophils, eosinophils, monocytes), megakaryocytes, synovial cells, chondrocytes, osteocytes, osteoblasts, osteoclasts, mammary cells, interstitial cells, or progenitor cells of these cells, stem cells or cancer cells, etc.) of human or other warm-blooded animals (e.g. guinea pig, rat, mouse, chicken, rabbit, pig, sheep, bovine, monkey, etc.) or all tissues in which such cells are present, such as brain, various parts of brain (e.g. olfactory bulb, amygdaloid nucleus, cerebral basal nucleus, hippocampus, thalamus, hypothalamus, cerebral cortex, medulla oblongata, cerebellum), spinal cord, pituitary gland, stomach, pancreas, kidney, liver, gonad, thyroid, gall-bladder, bone marrow, adrenal gland, skin, muscle, lung, gastrointestinal tracts (e.g. large intestine, small intestine), blood vessels, heart, thymus, spleen, salivary gland, peripheral blood, prostate, testis, ovary, placenta, uterus, bone, cartilage, joint, skeletal muscle, etc. It may also be a recombinant peptide or a synthetic peptide.

The term “substantially identical” has the same meaning as described above in the explanation of the polypeptide of the invention.

An amino acid sequence substantially identical with the amino acid sequence as shown in SEQ ID NO: 6 or SEQ ID NO: 8 is not particularly limited as long as the peptide comprising the relevant amino acid sequence has an activity (nature) substantially identical with the activity of a polypeptide comprising the amino acid sequence as shown in SEQ ID NO: 6 or SEQ ID NO: 8. For example, an amino acid sequence having about 70% or more, preferably about 80% or more, more preferably about 90% or more, still more preferably about 95% or more and most preferably about 98% or more homology to the amino acid sequence as shown in SEQ ID NO: 6 or SEQ ID NO: 8 may be used. However, as an amino acid sequence substantially identical with the amino acid sequence as shown in SEQ ID NO: 6, a sequence where the cysteine residues of SEQ ID NO: 6 are conserved is preferable.

More specifically, the amino acid sequence as shown in SEQ ID NO: 6 or the amino acid sequence as shown in SEQ ID NO: 8 may be used, for example.

With respect to the activity (nature) of substantially the same quality mentioned above, this means that, for example, the effect of preventing and/or treating cancers, immunological diseases, infections, gastrointestinal diseases, circulatory organ diseases, endocrine diseases and fertility; the effect of regulating the differentiation and/or function of germ cells; or the effect of regulating the differentiation and/or proliferation of blood cells possessed by the peptide comprising the amino acid sequence as shown in SEQ ID NO: 6 or SEQ ID NO: 8 is qualitatively identical.

More specific examples of peptides comprising the amino acid sequence as shown in SEQ ID NO: 6 or SEQ ID NO: 8 include the so-called muteins, such as (1) peptides comprising (i) the amino acid sequence of SEQ ID NO: 6 wherein one or two or more amino acids (e.g. about 1-10, preferably about 1-5, more preferably 1 or 2 amino acids) are deleted therefrom; (ii) the amino acid sequence of SEQ ID NO: 6 wherein one or two or more amino acids (e.g. about 1-10, preferably about 1-5, more preferably 1 or 2 amino acids) are added thereto; (iii) the amino acid sequence of SEQ ID NO: 6 wherein one or two or more amino acids (e.g. about 1-10, preferably about 1-5, more preferably 1 or 2 amino acids) are replaced with other amino acids; or (iv) an amino acid sequence which is a combination of these sequences and (2) peptides comprising (i) the amino acid sequence of SEQ ID NO: 8 wherein one or two or more amino acids (e.g. about 1-5, preferably 1 or 2 amino acids) are deleted therefrom; (ii) the amino acid sequence of SEQ ID NO: 8 wherein one or two or more amino acids (e.g. about 1-5, preferably 1 or 2 amino acids) are added thereto; (iii) the amino acid sequence of SEQ ID NO: 8 wherein one or two or more amino acids (e.g. about 1-5, preferably 1 or 2 amino acids) are replaced with other amino acids; or (iv) an amino acid sequence which is a combination of these sequences.

When the amino acid sequence is inserted, deleted or substituted as described above, the site of insertion, deletion or substitution is not particularly limited. In SEQ ID NO: 6 or SEQ ID NO: 8, however, positions other than the basic amino acid pairs and the cysteine residues in the amino acid sequence of SEQ ID NO: 6 may be preferable.

As a partial peptide of the polypeptide of the invention or the peptide of the invention (the partial peptide of the invention), any partial peptide of the above-described polypeptide or peptide of the invention may be used. However, a partial peptide having an activity of substantially the same quality as that of the polypeptide of the invention or the peptide of the invention is preferably used (the term “activity of substantially the same quality” has the same meaning as described above).

Different from the polypeptide of the invention or the peptide of the invention, the partial peptide of the invention may be used as antigen for preparing antibodies. Thus, the partial peptide of the invention does not necessarily need to have the activity possessed by the polypeptide of the invention or the peptide of the invention.

For example, a peptide comprising at least 5 or more, preferably 10 or more, amino acids from the amino acid sequence of the polypeptide of the invention may be used.

The partial peptide of the invention may have deletion of one or two or more amino acids (preferably about 1-10, more preferably several (1-5) amino acids) in its amino acid sequence; or addition of one or two or more amino acids (preferably about 1-20, more preferably 1-10, still more preferably several (1-5) amino acids) to its amino acid sequence; or insertion of one or two or more amino acids (preferably about 1-20, more preferably 1-10, still more preferably several (1-5) amino acids) into its amino acid sequence; or substitution of one or two or more amino acids (preferably about 1-10, more preferably several, still more preferably about 1-5 amino acids) with other amino acids in its amino acid sequence.

The polypeptide of the invention, the peptide of the invention and the partial peptide of the invention also encompass those polypeptides/peptides where substituents on side chains of intramolecular amino acids are protected with appropriate protective groups, or conjugated peptides such as glycopeptides to which sugar chains are attached.

Further, the polypeptide of the invention or the peptide of the invention may exist not only as a monomer but also as a dimer, a trimer, a tetramer, etc. Specifically, possible forms include, but are not limited to, the following: the polypeptide of the invention is a monomer; or two polypeptides of the invention form a dimer; or the peptide of the invention is a monomer; or two peptides of the invention form a dimer; or the polypeptide of the invention and the peptide of the invention form a dimer.

As shown especially in Example 4 described later, the polypeptide or the peptide of the invention may form a dimer linked by disulfide bonds through cysteine residues in SEQ ID NO: 4 and SEQ ID NO: 6. Also, a monomer may be formed as a result of formation of an intramolecular disulfide bond through cysteine residues in SEQ ID NO: 6. Specifically, possible structures of dimers which the polypeptide of the invention and the peptide of the invention can take include (i) a dimer composed of two polypeptides each consisting of the amino acid sequence as shown in SEQ ID NO: 4; (ii) a dimer composed of two peptides each consisting of the amino acid sequence as shown in SEQ ID NO: 6; and (iii) a dimer composed of a polypeptide consisting of the amino acid sequence of SEQ ID NO: 4 and a peptide consisting of the amino acid sequence of SEQ ID NO: 6. Possible structures of monomers which the polypeptide of the invention and the peptide of the invention can take include (i) a monomer of a polypeptide consisting of the amino acid sequence as shown in SEQ ID NO: 4 and (ii) a monomer of a peptide consisting of the amino acid sequence as shown in SEQ ID NO: 6.

Further, the polypeptide of the invention and the peptide of the invention may comprise any foreign sequence (for example, such as FLAG, His tag, HA tag or HSV tag) that could be an epitope (antibody recognition site) located at, for example, their N-terminal or C-terminal.

Specific examples of such (poly)peptides include a polypeptide comprising the amino acid sequence as shown in SEQ ID NO: 14.

The above-described dimers may be prepared by linking monomers by chemical techniques or may be produced by conventional genetic engineering techniques.

The polypeptide, the peptide and the partial peptide of the invention are expressed in accordance with the conventions for description of peptides, that is, the N-terminus (amino terminus) at the left end and the C-terminus (carboxyl terminus) at the right end. The C-terminus of the polypeptide, the peptide or the partial peptide of the invention (such as a polypeptide comprising the amino acid sequence as shown in SEQ ID NO: 4) is usually a carboxyl group (—COOH) or a carboxylate (—COO⁻), but it may be an amide (—CONH₂) or an ester (—COOR).

Examples of R of the above ester group include C₁₋₆ alkyl groups (e.g. methyl, ethyl, n-propyl, isopropyl or n-butyl), C₃₋₈ cycloalkyl groups (e.g. cyclopentyl or cyclohexyl), C₆₋₁₂ aryl groups (e.g. phenyl or -naphthyl), C₇₋₁₄ aralkyl groups such as phenyl-C₁₋₂ alkyl groups (e.g. benzyl or phenethyl) and -naphthyl-C₁₋₂ alkyl groups (e.g. -naphthylmethyl). In addition, the ester group also includes pivaloyloxymethyl esters that are universally used as oral esters.

When the polypeptide or the partial peptide of the invention has a carboxyl group (or carboxylate) at any position other than its C-terminus, the carboxyl group may be amidated or esterified; such a polypeptide or partial peptide is also included in the polypeptide or the partial peptide of the invention. The ester in this case may be, for example, any of the esters mentioned above for the C-terminal ester.

Furthermore, the polypeptide, the peptide or the partial peptide of the invention includes those polypeptides, peptides or partial peptides in which the N-terminal amino acid residue (e.g. Met) is protected by a protective group (e.g. C₁₋₆ acyl group such as C₁₋₆ alkanoyl group (e.g. formyl group or acetyl group)); those polypeptides in which the N-terminal glutamine residue generated through in vivo cleavage is pyroglutaminated; those polypeptides, peptides or partial peptides in which a substituent on a side chain of an amino acid (e.g. —OH, —SH, amino group, imidazole group, indole group, or guannidino group) is protected by an appropriate protective group (e.g. C₁₋₆ acyl group such as C₁₋₆ alkanoyl group (e.g. formyl group or acetyl group)); and conjugated polypeptides such as the so-called glycopolypeptides to which sugar chains are linked.

As the salt of the polypeptide, the peptide or the partial peptide of the invention, salts formed with physiologically acceptable acids (e.g. organic or inorganic acids) or bases (e.g. alkali metals) are used. Especially preferable are physiologically acceptable acid addition salts. Examples of such salts include salts formed with inorganic acids (e.g. hydrochloric acid, phosphoric acid, hydrobromic acid or sulfuric acid) and salts formed with organic acids (e.g. acetic acid, formic acid, propionic acid, fumaric acid, maleic acid, succinic acid, tartaric acid, citric acid, malic acid, oxalic acid, benzoic acid, methanesulfonic acid or benzenesulfonic acid).

The polypeptide, the peptide or the partial peptide of the invention can be produced from the afore-mentioned cells or tissues of human or other warm-blooded animals by known purification methods for polypeptides (proteins). Alternatively, the polypeptide, the peptide or the partial peptide of the invention can be produced by culturing a transformant comprising the DNA of the invention described later encoding the polypeptide or the partial peptide of the invention. It can also be produced in accordance with the procedures for peptide synthesis which are described later.

When the polypeptide, the peptide or the partial peptide of the invention is produced from tissues or cells of human or mammals, the relevant tissue or cell is homogenized and then the polypeptide of the present invention is extracted with acids, etc. The polypeptide, the peptide or the partial peptide can be purified and isolated from the resultant extract by a combination of chromatography, such as reversed phase chromatography, ion exchange chromatography and so on.

For the synthesis of the polypeptide, the peptide or the partial peptide of the invention or a salt or amide thereof, any of the commercial resins available for polypeptide synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenylacetamidomethyl resin, polyacrylamide resin, 4-(2′,4′-dimethoxyphenyl-hydroxymethyl)phenoxy resin, and 4-(2′,4′-dimethoxyphenyl-Fmoc-aminoethyl)phenoxy resin. Using such a resin, amino acids protected at their -amino groups and side chain functional groups are condensed on the resin according to the amino acid sequence of the polypeptide of interest by conventional condensation methods. At the final stage of the reaction, all protective groups are removed simultaneously with the cleavage of the polypeptide from the resin. Then, in a highly diluted solution, intramolecular disulfide bond formation reaction is carried out to obtain the polypeptide of interest or amide thereof.

With respect to condensation of the above-described protected amino acids, various activators useful for polypeptide synthesis may be utilized. Among all, carbodiimide reagents are especially preferred. Examples of carbodiimide reagents include DCC, N,N′-diisopropylcarbodiimide, and N-ethyl-N′-(3-dimethylaminoprolyl)carbodiimide. For activation by these reagents, protected amino acids and a recemization inhibitor (e.g. HOBt or HOOBt) may be directly added to the resin, or protected amino acids may be activated in advance in the form of symmetric acid anhydride, HOBt ester or HOOBt ester and then added to the resin.

The solvent used for the above-mentioned activation of protected amino acids or the condensation thereof with a resin may be appropriately selected from those solvents known to be useful for polypeptide (protein) condensation reactions. Examples of useful solvents include acid amides (e.g. N,N-dimethylformamide, N,N-dimethylacetamide or N-methylpyrrolidone), halogenated hydrocarbons (e.g. methylene chloride, or chloroform), alcohols (e.g. trifluoroethanol), sulfoxides (e.g. dimethyl sulfoxide), ethers (e.g. pyridine, dioxane, tetrahydrofuran), nitriles (e.g. acetonitrile or propionitrile), esters (e.g. methyl acetate or ethyl acetate), and suitable mixtures of these solvents. The reaction temperature may be appropriately selected from the range known to be useful for polypeptide (protein) bond-forming reactions; usually, the temperature is selected from the range from about −20 C to about 50 C. The activated amino acid derivative is usually used in 1.5- to 4-fold excess. When the condensation is found insufficient as a result of test using the ninhydrin reaction, sufficient condensation can be achieved by repeating reactions without removing protective groups. When sufficient condensation cannot be achieved even by repeating reactions, unreacted amino acids may be acetylated with acetic anhydride or acetylimidazole so that they do not affect subsequent reactions.

Examples of useful protective groups for the amino group of raw materials include Z, Boc, t-pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulfenyl, diphenylphosphinothioyl, and Fmoc.

The carboxyl group can be protected, for example, in the form of an alkyl ester (e.g. straight-chain, branched, or cyclic alkyl esters such as methyl, ethyl, propyl, butyl, t-butyl, cyclopentyl, cyclohexyl, cycloheptyl, cyclooctyl, 2-adamantyl, and so on), aralkyl ester (e.g. benzyl ester, 4-nitrobenzyl ester, 4-methoxybenzyl ester, 4-chlorobenzyl ester, benzhydryl ester, and so on), phenacyl ester, benzyloxycarbonylhydrazide, t-butoxycarbonylhydrazide or tritylhydrazide.

The hydroxyl group of serine can be protected, for example, by esterification or etherification. Examples of suitable groups for this esterification include lower (C₁₋₆) alkanoyl groups such as acetyl, aroyl groups such as benzoyl, and carbonic acid-derived groups such as benzyloxycarbonyl and ethyloxycarbonyl. Examples of groups suitable for the etherification include benzyl, tetrahydropyranyl and t-butyl.

Examples of protective groups for the phenolic hydroxyl group of tyrosine include Bzl, Cl₂-Bzl, 2-nitrobenzyl, Br-Z, and t-butyl.

Examples of protective groups for the imidazole ring of histidine include Tos, 4-methoxy-2,3,6-trimethylbenzenesulfonyl, DNP, benzyloxymethyl, Bum, Boc, Trt and Fmoc.

Examples of raw materials with activated carboxyl groups include the corresponding acid anhydrides, azides and active esters (esters of alcohols such as pentachlorophenol, 2,4,5-trichlorophenol, 2,4-dinitrophenol, cyanomethyl alcohol, p-nitrophenol, HONB, N-hydroxysuccinimide, N-hydroxyphthalimide and HOBt). Examples of raw materials with activated amino groups include the corresponding phosphoric acid amides.

Methods for removing (eliminating) protective groups include, for example, catalytic reduction in a hydrogen stream in the presence of a catalyst such as Pd-black or Pd-carbon, acid treatment with anhydrous hydrogen fluoride, methanesulfonic acid, trifluoromethanesulfonic acid, trifluoroacetic acid or mixtures thereof, treatment with a base such as diiso-propylethylamine, triethylamine, piperidine, piperazine or the like, and reduction with sodium in liquid ammonia. The elimination reaction by the above-mentioned acid treatment is generally conducted at temperatures of about −20 C to about 40 C. In the acid treatment, it is effective to add a cation scavenger such as anisole, phenol, thioanisole, m-cresol, p-cresol, dimethylsulfide, 1,4-butanedithiol or 1,2-ethanedithiol. The 2,4-dinitrophenyl group used as the protective group for the imidazole ring of histidine is removed by thiophenol treatment. The formyl group used as the protective group for the indole ring of tryptophan may be removed by the above-mentioned deprotection by the acid treatment in the presence of 1,2-ethanedithiol, 1,4-butanedithiol or the like, or by alkali treatment using dilute sodium hydroxide, dilute ammonia or the like.

The protection of functional groups in raw materials that should not be involved in the reaction, protective groups therefor, the removal of these protective groups and the activation of functional groups involved in the reaction can be appropriately selected from groups or methods known in the art.

An alternative method for obtaining amides of the polypeptide, the peptide or partial peptide of the invention comprises, for example, protecting the -carboxyl group of the C-terminal amino acid by amidation, extending the peptide (polypeptide) chain to a desired length on the side of the amino group, preparing a polypeptide with its N-terminal -amino group selectively deprotected, preparing a polypeptide with its C-terminal carboxyl group selectively deprotected, and condensing these two polypeptides in a mixed solvent such as described above. Details of this condensation reaction are the same as described above. After purification of the protected polypeptide thus obtained by condensation, all the protective groups are removed by the method described above to thereby to provide a crude polypeptide of interest. This crude polypeptide is purified by various known purification techniques and lyophilized to provide the desired polypeptide, peptide or partial peptide in an amide form.

A method for obtaining esters of the polypeptide, the peptide or the partial peptide of the invention, for example, condensing the -carboxyl group of the C-terminal amino acid with a desired alcohol to prepare the corresponding amino acid ester, and subjecting this ester to the same procedures as described above in the preparation of amides to thereby provide the desired polypeptide, peptide or partial peptide in an ester form.

The polypeptide, the peptide or the partial peptide of the invention can be produced by known methods for peptide synthesis. The method for peptide synthesis may be solid-phase synthesis or liquid-phase synthesis. Briefly, a peptide of interest can be produced by condensing a partial peptide or amino acid capable of constituting the partial peptide of the invention with the residual part thereof and, if the product has protective groups, removing the protective groups. Examples of condensation methods and methods for removal of protective groups known in the art include those described in the following references (i) to (v).

(i) M. Bodanszky & M. A. Ondetti, Peptide Synthesis, Interscience Publishers, New York, 1966

(ii) Schroeder & Luebke, The Peptide, Academic Press, New York, 1965

(iii) Nobuo Izumiya et al., Fundamentals and Experiments in Peptide Synthesis, Maruzen, 1975

(iv) Haruaki Yajima and Shumpei Sakakibara, Biochemical Experiment Series 1, Polypeptide Chemistry IV, 205, 1977, and

(v) Haruaki Yajima (ed.), Development of Drugs (Continued), Vol. 14, Peptide Synthesis, Hirokawa Shoten

After the reaction, the polypeptide or the partial peptide of the invention can be isolated and purified by a combination of conventional purification techniques such as solvent extraction, distillation, column chromatography, liquid chromatography, and recrystallization. When the polypeptide thus obtained is a free polypeptide, it can be converted to a suitable salt by known methods or methods based thereon. On the contrary, when the polypeptide is obtained in a salt form, it can be converted to a free polypeptide or another salt by known methods or methods based thereon.

The polynucleotide encoding the polypeptide, the peptide or the partial peptide of the invention (hereinafter, such polynucleotide may be referred to as the “polynucleotide of the invention” collectively) may be any polynucleotide as long as it comprises a nucleotide sequence encoding the above-described polypeptide, peptide or partial peptide of the invention. Preferably, the polynucleotide is DNA (hereinafter, sometimes referred to as the “DNA of the invention” collectively). The polynucleotide may be genomic DNA, cDNA derived from the above-mentioned cells or tissues, or synthetic DNA.

Vectors used for library construction may be any vectors such as bacteriophage, plasmid, cosmid, phagemid, and so on. Alternatively, total RNA or mRNA fraction may be prepared from the above-mentioned cells or tissues, followed by direct amplification by reverse transcriptase polymerase chain reaction (hereinafter, abbreviated to as “RT-PCR”).

With respect to a polynucleotide encoding the polypeptide of the invention, the polynucleotide may be any polynucleotide as long as it comprises a polynucleotide encoding a polypeptide having an activity (nature) of substantially the same quality as that of the polypeptide of the invention [e.g. immunogenicity; activity (effect) of preventing and/or treating cancers, immunological diseases, infections, gastrointestinal diseases, circulatory organ diseases, endocrine diseases and fertility; the activity (effect) of regulating the differentiation and/or function of germ cells; or the activity (effect) of regulating the differentiation and/or proliferation of blood cells] and yet encodes a polypeptide having a nature of substantially the same quality as that of the polypeptide of the invention.

Specific examples of the polynucleotide encoding the polypeptide of the invention include:

(i) polynucleotides comprising a polynucleotide having the nucleotide sequence as shown in SEQ ID NO: 5,

(ii) polynucleotides comprising a polynucleotide having the nucleotide sequence as shown in SEQ ID NO: 3,

(iii) polynucleotides comprising a polynucleotide having the nucleotide sequence as shown in SEQ ID NO: 1, and

(iv) polynucleotides comprising a polynucleotide having the nucleotide sequence as shown in SEQ ID NO: 15. Of these, “polynucleotides comprising a polynucleotide having the nucleotide sequence as shown in SEQ ID NO: 3”, “polynucleotides comprising a polynucleotide having the nucleotide sequence as shown in SEQ ID NO: 1” and “polynucleotides comprising a polynucleotide having the nucleotide sequence as shown in SEQ ID NO: 15” are included in specific examples of “polynucleotides comprising a polynucleotide having the nucleotide sequence as shown in SEQ ID NO: 5”.

Nucleotides capable of hybridizing to the nucleotide sequence as shown in SEQ ID NO: 5, SEQ ID NO: 1, SEQ ID NO: 3 or SEQ ID NO: 15 under high stringency conditions may also be enumerated as nucleotides encoding the polypeptide of the invention. For example, polynucleotides comprising a nucleotide sequence having about 70% or more, preferably about 80% or more, still more preferably about 90% or more homology to the nucleotide sequence as shown in SEQ ID NO: 5, SEQ ID NO: 1, SEQ ID NO: 3 or SEQ ID NO: 15 may be used.

Specific examples of nucleotides capable of hybridizing to the nucleotide sequence as shown in SEQ ID NO: 5, SEQ ID NO: 1, SEQ ID NO: 3 or SEQ ID NO: 15 under high stringency conditions include polynucleotides that have a nucleotide sequence hybridizing to the nucleotide sequence as shown in SEQ ID NO: 5, SEQ ID NO: 1, SEQ ID NO: 3 or SEQ ID NO: 15 under high stringency conditions and encode a polypeptide having a nature of substantially the same quality (the meaning of this term is as described above) as that of the polypeptide of the invention.

Hybridization can be carried out according to known methods or methods based thereon, e.g. those methods described in Molecular Cloning, 2nd Ed. (J. Sambrook et al., Cold Spring Harbor Lab. Press, 1989). When commercial libraries are used, hybridization can be carried out in accordance with the methods described in the instructions attached thereto; more preferably, hybridization is carried out under high stringency conditions.

“High stringency conditions” refers to, for example, conditions where sodium concentration is about 19-40 mM, preferably about 19-20 mM, and temperature is about 50-70 C, preferably about 60-65 C.

As a polynucleotide encoding the polypeptide of the invention having the amino acid sequence as shown in SEQ ID NO: 4, a polynucleotide having the nucleotide sequence as shown in SEQ ID NO: 5 may be used, for example. As a polynucleotide encoding the polypeptide of the invention having the amino acid sequence as shown in SEQ ID NO: 2, a polynucleotide having the nucleotide sequence as shown in SEQ ID NO: 3 may be used, for example. As a DNA encoding the polypeptide of the invention having the amino acid sequence as shown in SEQ ID NO: 14, a polynucleotide having the nucleotide sequence as shown in SEQ ID NO: 15 may be used, for example. A polynucleotide having the nucleotide sequence as shown in SEQ ID NO: 1 is, as described in Example 1, a DNA comprising a polynucleotide having the nucleotide sequence as shown in SEQ ID NO: 3 as a polynucleotide encoding the polypeptide of the invention having the amino acid sequence as shown in SEQ ID NO: 2.

With respect to a polynucleotide encoding the peptide of the invention, the polynucleotide may be any polynucleotide as long as it encodes the peptide of the invention.

Specific examples of polynucleotides encoding the peptide of the invention include:

(i) polynucleotide comprising a polynucleotide having the nucleotide sequence as shown in SEQ ID NO: 7, and

(ii) polynucleotide comprising a polynucleotide having the nucleotide sequence as shown in SEQ ID NO: 9.

Nucleotides capable of hybridizing to the nucleotide sequence as shown in SEQ ID NO: 7 or SEQ ID NO: 9 under high stringency conditions may also be enumerated as nucleotides encoding the partial peptide of the invention. For example, polynucleotides comprising a nucleotide sequence having about 70% or more, preferably about 80% or more, still more preferably about 90% or more homology to the nucleotide sequence as shown in SEQ ID NO: 7 or SEQ ID NO: 9 may be used.

Hybridization can be carried out according to known methods or methods based thereon, e.g. those methods described in Molecular Cloning, 2nd Ed. (J. Sambrook et al., Cold Spring Harbor Lab. Press, 1989). When commercial libraries are used, hybridization can be carried out in accordance with the methods described in the instructions attached thereto; more preferably, hybridization is carried out under high stringency conditions.

“High stringency conditions” refers to, for example, conditions where sodium concentration is about 19-40 mM, preferably about 19-20 mM, and temperature is about 50-70 C, preferably about 60-65 C.

As a polynucleotide encoding the peptide of the invention having the amino acid sequence as shown in SEQ ID NO: 6, a polynucleotide having the nucleotide sequence as shown in SEQ ID NO: 7 may be used, for example. As a polynucleotide encoding the peptide of the invention having the amino acid sequence as shown in SEQ ID NO: 8, a polynucleotide having the nucleotide sequence as shown in SEQ ID NO: 9 may be used, for example.

With respect to a polynucleotide encoding the partial peptide of the invention, the polynucleotide may be any polynucleotide as long as it encodes the partial peptide of the invention.

The cloning of a DNA encoding the full length of the polypeptide of the invention, the peptide of the invention (hereinafter, sometimes the polypeptide and peptide of the invention are referred to as the “polypeptide of the invention” collectively) or the partial peptide of the invention can be cloned either by PCR amplification from genomic DNA or cDNA using synthetic DNA primers each having a partial nucleotide sequence of the polypeptide or partial peptide of the invention, or by a method where a DNA fragment of interest is selected by hybridizing DNA inserted into an appropriate vector (i.e. library) to a DNA probe labeled with a radio isotope or enzyme, the DNA probe being a DNA fragment or a synthetic DNA encoding a part or full length of the polypeptide or partial peptide of the invention; then, the selected DNA fragment is integrated into a recombinant vector, and a host such as E. coli is transformed with the resultant vector. The hybridization can be carried out, for example, according to the method described in Molecular Cloning, 2nd Edition (J. Sambrook et al., Cold Spring Harbor Lab. Press, 1989). When commercial libraries are used, the hybridization can be carried out according to the instructions attached thereto.

Substitution of the nucleotide sequence of a DNA can be performed by known methods such as ODA-LA PCR, the gapped duplex method, the Kunkel method and the like using PCR, known kits such as Mutan™-Super Express Km (Takara Shuzo), Mutan™-K (Takara Shuzo), etc.

The cloned DNA encoding the polypeptide of the invention may be used as it is or after digestion with restriction enzymes or addition of linkers, depending on purposes. The DNA may have ATG at its 5′ end as a translation initiation codon and TAA, TGA, or TAG at its 3′ end as a translation termination codon. The translation initiation and termination codons may also be added by using appropriate synthetic DNA adapters.

Expression vectors for the polypeptide for the partial peptide of the invention can be prepared by, for example, (a) cutting out a DNA fragment of interest from a DNA (e.g. cDNA) encoding the polypeptide or the partial peptide of the invention and (b) ligating the DNA fragment to an appropriate expression vector downstream of its promoter.

Examples of vectors include plasmids derived from Escherichia coli (e.g. pBR322, pBR325, pUC12, and pUC13); plasmids derived from Bacillus subtilis (e.g. pUB110, pTP5 and pC194); plasmids derived from yeast (e.g. pSH19 and pSH15); bacteriophages such as -phage; animal viruses such as retrovirus, vaccinia virus, baculovirus; and other vectors such as pA 1-11, pXT1, pRc/CMV, pRc/RSV, pcDNAI/Neo and so on.

Any promoter may be used in the invention as long as it is appropriate for the host that will be used for expressing a gene of interest. When the host is an animal cell, examples of promoters include SR promoter, SV40 promoter, LTR promoter, CMV promoter, HSV-TK promoter and -actin promoter or the like.

Among these promoters, CMV (cytomegalovirus) promoter, SR promoter or the like is preferably used. When the host is an Escherichia bacterium, trp promoter, lac promoter, recA promoter, P_{L} promoter, lpp promoter, T7 promoter or the like is preferably used. When the host is a Bacillus bacterium, SPO1 promoter, SPO₂ promoter, penP promoter or the like is preferably used. When the host is a yeast, PHO5 promoter, PGK promoter, GAP promoter, ADH promoter, or the like is preferably used. When the host is an insect cell, polyhedrin promoter, P10 promoter or the like is preferably used.

The expression vectors may, if desired, further comprise enhancers, splicing signals, polyadenylation signals, selective markers, SV40 replication origin (hereinafter, sometimes abbreviated to “SV40 ori”) and the like. Examples of selective markers include dihydrofolate reductase (hereinafter, sometimes abbreviated to “dhfr”) gene [methotorexate (MTX) resistance], ampicillin resistance gene (hereinafter, sometimes abbreviated to “Amp^{r}”), neomycin resistance gene [hereinafter, sometimes abbreviated to “Neo^{r}”: Geneticin (G418) resistance] and the like. When dhfr gene-deficient Chinese hamster cells are used in combination with dhfr gene as a selective marker, recombinant cells may be selected even in a thymidine-free medium.

Furthermore, a signal sequence appropriate for the host may be added, if necessary, to the N-terminal of the polypeptide of the invention. When the host is an Escherichia bacterium, PhoA signal sequence, OmpA signal sequence or the like may be used. When the host is a Bacillus bacterium, -amylase signal sequence, subtilisin signal sequence or the like may be used. When the host is yeast, MF signal sequence, SUC2 signal sequence or the like may be used. When the host is an animal cell, insulin signal sequence, -interferon signal sequence, antibody molecule signal sequence or the like may be used.

Using the thus constructed vector comprising a DNA encoding the polypeptide or the partial peptide of the invention, transformants can be prepared.

Examples of hosts include bacteria belonging to the genus Escherichia, bacteria belonging to the genus Bacillus, yeasts, insect cells, insects, and animal cells.

Specific examples of bacteria belonging to the genus Escherichia include E. coli K12 DH1 [Proc. Natl. Acad. Sci. USA, Vol. 60, 160 (1968)], JM103 [Nucleic Acids Research, Vol. 9, 309 (1981)], JA221 [Journal of Molecular Biology, Vol. 120, 517 (1978)]), HB101 [Journal of Molecular Biology, Vol, 41, 459 (1969)] and C600 [Genetics, Vol. 39, 440 (1954)].

Specific examples of bacteria belonging to the genus Bacillus include B. subtilis MI114 [Gene, Vol. 24, 255 (1983)] and 207-21 [Journal of Biochemistry, Vol. 95, 87 (1984)].

Specific examples of yeasts include Saccharomyces cerevisiae AH22, AH22R, NA87-11A, DKD-5D and 20B-12, Schizosaccharomyces pombe NCYC1913 and NCYC2036, and Pichia pastoris KM71.

Specific examples of insect cells include, when the virus used is AcNPV, a cell line derived from larvae of Spodoptera frugiperda (Sf cells), MG1 cells derived from the midgut of Trichoplusia ni, High Five™ cells derived from eggs of Trichoplusia ni, Mamestra brassicae-derived cells and Estigmena acrea-derived cells. When the virus used is BmNPV, insect cells such as a silkworm-derived cell line (Bombyx mori N cells; BmN cells) may be used. Specific examples of Sf cells include Sf9 cells (ATCC CRL 1711) and Sf21 cells [both disclosed in Vaughn J. L. et al., In Vivo, 13, 213-217 (1977)].

Specific examples of insects include larvae of silkworm (Maeda et al., Nature, 315, 592 (1985)).

Specific examples of animal cells include simian cell COS-7, Vero cells, Chinese hamster cell CHO (hereinafter, abbreviated to “CHO cells”), dhfr gene-deficient Chinese hamster cell CHO (hereinafter, abbreviated to “CHO(dhfr⁻) cells”), mouse L cells, mouse AtT-20 cells, mouse myeloma cells, rat GH3 cells, and human FL cells.

Transformation of bacteria belonging to the genus Escherichia can be performed in accordance with methods disclosed, for example, in Proc. Natl. Acad. Sci. USA, Vol. 69, 2110 (1972) and Gene, Vol. 17, 107 (1982).

Transformation of bacteria belonging to the genus Bacillus can be performed in accordance with methods disclosed, for example, in Molecular & General Genetics, Vol. 168, 111 (1979).

Transformation of yeasts can be performed in accordance with methods disclosed, for example, in Methods in Enzymology, 194, 182-187(1991) and Proc. Natl. Acad. Sci. USA, Vo. 75, 1929 (1978).

Transformation of insect cells or insects can be performed in accordance with methods disclosed, for example, in Bio/Technology, 6, 47-55 (1988).

Transformation of animal cells can be performed by methods disclosed, for example, in Cell Engineering, Separate Vol. 8, New Cell Engineering Experiment Protocol, 263-267 (1995) (Shujunsha Co.) and Virology, Vol. 52, 456 (1973).

Thus, transformants transformed with the expression vector comprising a DNA encoding the polypeptide can be obtained.

As a medium to culture transformants obtained from Escherichia or Bacillus bacteria as hosts, a liquid medium is appropriate. The medium may contain carbon sources, nitrogen sources, minerals, and so on which are necessary for the growth of the transformant. As carbon sources, glucose, dextrin, soluble starch, sucrose or the like may be enumerated. As nitrogen sources, organic or inorganic substances such as ammonium salts, nitrates, corn steep liquor, peptone, casein, yeast extract, meat extract, bean cake, potato extract, or the like may be enumerated. As minerals, calcium chloride, sodium dihydrogen phosphate, magnesium chloride, or the like may be enumerated. Further, yeast, vitamins, growth-promoting factors, etc. may also be added to the medium. Preferable pH of the medium is about 5-8.

As a medium to culture Escherichia bacteria, M9 medium containing glucose and casamino acid [Miller, Journal of Experiments in Molecular Genetics, 431-433, Cold Spring Harbor Laboratory, New York, (1972)] is preferable, for example. If necessary, drugs such as 3-indolyl acrylic acid can be added to the medium to improve the efficiency of the promoter. When the host is an Escherichia bacterium, the transformant is cultured usually at about 15-43 C for about 3-24 hours. If necessary, aeration and stirring may be applied.

When the host is a Bacillus bacterium, the transformant is cultured usually at about 30-40 C for about 6-24 hours. If necessary, aeration and stirring may also be applied.

As a medium to culture transformants obtained from yeasts as hosts, a medium such as Burkholder minimum medium [Bostian, K. L. et al., Proc. Natl. Acad. Sci. USA, Vol. 77, 4505 (1980)] or SD medium containing 0.5% casamino acid [Bitter, G. A. et al., Proc. Natl. Acad. Sci. USA, Vol. 81, 5330 (1984)] may be used, for example. It is preferable that the pH of the medium be adjusted to about 5-8. The transformant is cultured usually at about 20-35 C for about 24-72 hours. If necessary, aeration and stirring may be applied.

As a medium to culture transformants obtained from insect cells or insects as hosts, Grace's Insect Medium [Grace, T. C. C., Nature, 195, 788 (1962)] supplemented with additives such as inactivated 10% bovine serum may be used, for example. It is preferable that the pH of the medium be adjusted to about 6.2-6.4. The transformant is cultured usually at about 27 C for about 3-5 days. If necessary, aeration and stirring may be applied.

As a medium to culture transformants obtained from animal cells as hosts, examples of media include MEM medium [Science, Vol. 122, 501 (1952)], DMEM medium [Virology, Vol. 8, 396 (1959)], RPMI 1640 medium [Journal of the American Medical Association, Vol. 199, 519 (1967)] and 199 medium [Proceedings of the Society for the Biological Medicine, Vol. 73, 1 (1950)] each containing about 5-20% fetal calf serum. Preferable pH of the medium is from about 6 to about 8. The transformant is cultured usually at about 30-40 C for about 15-60 hours. If necessary, aeration and stirring may be applied.

Thus, it is possible to allow the transformant to produce the polypeptide or the partial peptide of the invention within cells or cell membranes, or out of cells (preferably out of cells).

Separation and purification of the polypeptide or the partial peptide of the invention from the resultant culture can be carried out, for example, according to the methods described below.

For extraction of the polypeptide or the partial peptide of the invention from cultured microorganisms or cells, the microorganism cells are harvested by known methods after the cultivation, suspended in a suitable buffer, and disrupted by sonication or by lysozyme and/or freezing and thawing, etc. Then, a crude extract of the polypeptide extract is obtained by centrifugation or filtration. The buffer may contain a protein denaturing agent such as urea or guanidine hydrochloride, or a surfactant such as Triton X100™. If the protein of interest is secreted into the culture broth, the supernatant is separated from the microorganisms or cells after completion of the cultivation and collected by known methods.

Purification of the polypeptide or the partial peptide of the invention contained in the resultant culture supernatant or extract can be performed by an appropriate combination of known methods for separation and purification. These known methods include methods utilizing solubility (such as salting out or sedimentation with solvents), methods mainly utilizing difference in molecular weight (such as dialysis, ultrafiltration, gel filtration and SDS-polyacrylamide gel electrophoresis), methods utilizing difference in electric charge (such as ion-exchange chromatography), methods utilizing specific affinity (such as affinity chromatography), methods utilizing difference in the hydrophobicity (such as reversed-phase high-performance liquid chromatography), and methods utilizing difference in isoelectric point (such as isoelectric electrophoresis).

When the thus obtained polypeptide or partial peptide of the invention is a free form, it can be converted into the above-described salt by known methods or methods based thereon. On the contrary, when the protein of interest is obtained in a salt form, the salt can be converted into a free form or another salt according to known methods or methods based thereon.

The protein of interest produced by the transformant can be arbitrarily modified or a part thereof can be removed therefrom by using an appropriate protein modification enzyme or protease before or after the purification. Examples of such enzymes include trypsin, chymotrypsin, arginyl endopeptidase, protein kinase and glycosidase.

The presence of the thus produce polypeptide or partial peptide of the invention can be measured by enzyme immunoassays, Western blot analysis, etc. using specific antibodies.

Alternatively, the presence of the polypeptide of the invention can be measured by fusing any foreign peptide sequence (e.g. FLAG, HIS tag, myc tag, HA tag, or HSV tag) that could be an epitope (antibody recognition site) to the N-terminal, C-terminal, etc. of the polypeptide as described earlier and then detecting chemiluminescence or the like using an antibody that recognizes the above peptide sequence. For example, a polypeptide comprising the amino acid sequence as shown in SEQ ID NO: 14 may be prepared and then the presence of the polypeptide of the invention may be measured by the method as described in Example 4 or the like.

An antibody to the polypeptide or the partial peptide of the invention (hereinafter, sometimes referred to as the “antibody of the invention”) may be either a polyclonal antibody (hereinafter, sometimes referred to as the “polyclonal antibody of the invention”) or a monoclonal antibody (hereinafter, sometimes referred to as the “monoclonal antibody of the invention”) as long as it can recognize the polypeptide or the partial peptide of the invention.

The antibody of the invention can be prepared using the polypeptide or the partial peptide of the invention as antigen and according to known methods for antibody or anti-serum preparation.

[Preparation of Monoclonal Antibodies]

(a) Preparation of Monoclonal Antibody-Producing Cells

The polypeptide or the partial peptide of the invention is administered to warm-blooded animals either alone or together with a carrier or diluent to a site capable of producing antibodies upon the administration. In order to enhance the ability to produce antibodies, complete Freund's adjuvants or incomplete Freund's adjuvants may also be administered. The administration is usually carried out once in every two to six weeks and two to ten times in the total. Examples of warm-blooded animals useful in the invention include monkey, rabbit, dog, guinea pig, mouse, rat, sheep, goat and chicken. Among them, mouse or rat is used preferably.

In the preparation of monoclonal antibody-producing cells, individuals with detectable antibody titers are selected from warm-blooded animals (e.g. mice) immunized with antigen. Then, the spleen or lymph nodes are collected from them two to five days after the final immunization, and antibody-producing cells contained therein are fused with myeloma cells of a homologous or heterologous animal to thereby obtain monoclonal antibody-producing hybridomas. Measurement of antibody titers in antisera may be carried out, for example, by reacting a labeled polypeptide (which will be described later) with the antiserum, followed by measuring the activity of the labeling agent bound to the antibody. The cell fusion may be carried out by a known method, for example, the method of Koehler and Milstein (Nature, 256, 495, (1975)). Examples of fusion promoters include polyethylene glycol (PEG), Sendai virus, etc. Preferably, PEG is used.

Examples of myeloma cells include myeloma cells of warm-blooded animals such as NS-1, P3U1, SP2/0, AP-1, etc. Preferably, P3U1 is used. A preferable ratio of the number of antibody-producing cells used (spleen cells) to the number of myeloma cells is from about 1:1 to about 20:1. When PEG (preferably, PEG 1000 to PEG 6000) is added at a concentration of about 10-80% and the resultant cell mixture is incubated at about 20-40 C (preferably, at about 30-37 C) for about 1-10 minutes, an efficient cell fusion can be achieved.

Various methods may be used for screening for monoclonal antibody-producing hybridomas. For example, hybridoma culture supernatant is added to a solid phase (e.g. microplate) on which the polypeptide antigen has been adsorbed either directly or with a carrier. Then, a radioactively or enzymatically labeled anti-immunoglobulin antibody (anti-mouse immunoglobulin antibody is used when mouse cells are used in the cell fusion) or protein A is added thereto to detect monoclonal antibodies bound to the solid phase. Alternatively, a method may be used in which hybridoma culture supernatant is added to a solid phase on which an anti-immunoglobulin antibody or protein A has been adsorbed; then, a radioactively or enzymatically labeled polypeptide is added thereto to thereby detect monoclonal antibodies bound to the solid phase.

Selection of monoclonal antibodies may be carried out by known methods or methods based on them. Usually, selection can be carried out in a medium for culturing animal cells supplemented with HAT (hypoxanthine, aminopterin and thymidine). As a medium for selection and culturing, any medium may be used as long as hybridomas are capable of growing therein. Examples of useful media include RPMI 1640 medium containing about 1-10% (preferably about 10-20%) of fetal calf serum, GIT medium (Wako Pure Chemical Industries, Ltd.) containing about 1-20% of fetal calf serum and a serum-free medium for hybridoma cultivation (SFM-101; Nissui Pharmaceutical Co.). The cultivation temperature is usually about 20-40 C, preferably about 37 C. The cultivation period is usually from five days to three weeks, preferably one to two weeks. The cultivation may be carried out usually under 5% carbon dioxide. The antibody titer of hybridoma culture supernatant may be measured in the same manner as in the above-mentioned measurement of the antibody titers in antisera.

(b) Purification of the Monoclonal Antibodies

Separation and purification of monoclonal antibodies may be carried out by conventional methods, such as methods for separating/purifying immunoglobulin [e.g. salting-out, alcohol precipitation, isoelectric precipitation, electrophoresis, adsorption/desorption using ion exchangers (e.g. DEAE), ultracentrifugation, gel filtration, specific purification methods in which only an antibody is collected by means of an antigen-binding solid phase or active adsorbent such as protein A or protein G, followed by dissociation of the bond].

[Preparation of Polyclonal Antibodies]

The polyclonal antibody of the invention can be produced by known methods or methods based on them. For example, an immunogen (antigen polypeptide) per se or a complex of the immunogen and a carrier protein is prepared. Then, using the immunogen or the complex, warm-blooded animals are immunized in the same manner as described for the production of monoclonal antibodies. Fractions containing the antibody against the polypeptide or the partial peptide of the invention are harvested from the immunized animals, followed by separation and purification of the antibody.

With respect to the immunogen-carrier protein conjugate for use in the immunization of warm-blooded animals, the kind of carrier protein and the mixing ratio of the carrier and the hapten are not particularly restricted as long as antibodies are produced efficiently against the hapten cross-linked to the carrier. For example, bovine serum albumin, bovine thyroglobulin, hemocyanin, or the like is coupled to the hapten at a weight ratio of about 0.1-20:1, preferably about 1-5:1.

A variety of condensing agents can be used for the coupling between the hapten and the carrier. For example, glutaraldehyde, carbodiimide, maleimide, or active ester reagents containing a thiol or dithiopyridyl group may be used.

The condensation product is administered to a warm-blooded animal either alone or together with a carrier or diluent at a site capable of producing antibodies. In order to enhance the antibody production ability upon the administration, complete Freund's adjuvant or incomplete Freund's adjuvant may also be administered. Administration is carried out generally once in about every 2-6 weeks and about 3-10 times in the total.

Polyclonal antibodies can be recovered from the blood, abdominal dropsy or other body fluid, preferably from the blood, of the warm-blooded animal immunized as described above.

Polyclonal antibody titers in antisera can be determined in the same manner as described above for the determination of monoclonal antibody titers in antisera. The separation and purification of polyclonal antibodies can be carried by the same methods for separation and purification of immunoglobulin as those described for the separation and purification of monoclonal antibodies.

With respect to the antisense DNA having a nucleotide sequence complementary to or substantially complementary to the DNA of the invention, any antisense DNA may be used as long as it has a nucleotide sequence complementary to or substantially complementary to the DNA of the invention and has an effect capable of inhibiting the expression of the DNA.

A nucleotide sequence substantially complementary to the DNA of the invention refers to, for example, a nucleotide sequence having about 70% or more, preferably about 80% or more, more preferably about 90% or more, most preferably about 95% or more homology to the full-length or a partial nucleotide sequence of the complementary nucleotide sequence to the DNA of the invention (i.e., the complementary strand to the DNA of the invention). Particularly preferable is an antisense DNA having about 70% or more, preferably about 80% or more, more preferably about 90% or more, most preferably about 95% or more homology to a part of the complementary strand to the DNA of the invention encoding an N-terminal portion of the polypeptide of the invention (e.g. nucleotide sequence encoding a region neighboring the initiation codon). These antisense DNAs can be synthesized with known DNA synthesizers.

When the polypeptide of the invention has a signal peptide, the peptide is efficiently secreted out of cells and manifests as a humoral factor important biological activities for signal transduction, self-defense, etc.

Hereinbelow, uses of the polypeptide of the invention, the partial peptide of the invention (sometimes, these two are collectively referred to as the “polypeptide of the invention”), the polynucleotide of the invention, the antibody of the invention and the antisense DNA will be described.

(1) As is clear from Example 2 described later, the polypeptide of the invention is expressed in tissues such as thymus, spleen, liver, pancreas, lymph nodes, lymphocytes, leukocytes and testis, among all, thymus and testis specifically. Thus, the polypeptide may be used as a tissue marker for these tissues. That is, this polypeptide is useful as a marker for detecting functions, disease state, cancer metastasis, infections, sterility, etc. of those tissues. Further, the polypeptide is also useful for obtaining corresponding receptors, binding polypeptides, etc. Moreover, the polypeptide may be used as a panel for high throughput screening to examine biological activities. The polypeptide may also be used in chromosomal mapping and utilized in investigations into genetic diseases.

(2) Therapeutic and/or Prophylactic for Various Diseases where the Polypeptide of the Invention is Involved

The polypeptide of the invention exists in vivo as a humoral factor. Therefore, when the polypeptide or the polynucleotide of the invention is abnormal, deficient, or expressed at an abnormally decreased or enhanced level, various diseases such as cancers, immunological diseases, infections, gastrointestinal diseases, circulatory organ diseases, endocrine diseases or sterility develop. When the polypeptide or the polynucleotide of the invention is abnormal, deficient, or expressed at an abnormally decreased or enhanced level, normal expression of functions of germ cells per se or sex hormone secretory cells is inhibited, which results in various disorders. Specifically, considering the fact that the polypeptide of the invention is expressed highly differentiated stage cells among male germ cells in the testis and that, in the epididymis, the polypeptide is highly expressed in the efferent ductules of the testis which lead sperm from the testis to the epididymis and in the head of the duct of epididymis, it is understood that the polypeptide of the invention possesses a physiological activity directly involved in the conservation of species (one of the most important abilities in biological species), such as the regulation of differentiation of sperm cells, the movement of sperm toward the epididymis, and the conferring of reproductive ability to sperm. Further, considering that the testis has Leydig cells producing a sex hormone (androgen) and thus is an important source of sex hormones, the polypeptide of the invention also possesses an effect of maintaining the normal function of Leydig cells.

Therefore, the polypeptide and the DNA of the invention may be used as medicines, for example, therapeutics and/or prophylactics for various diseases such as cancers, immunological diseases, infections (e.g. influenza, tuberculosis, gonococcal infection, viral hepatitis, vancomycin-resistant infection, methicillin-resistant infection, herpes, chlamydia, or sexually transmitted diseases (STD)), gastrointestinal diseases, circulatory organ diseases, endocrine diseases (e.g. abnormality in secondary sex characters, or feminization syndrome) or sterility (e.g. disroders of sperm production, epididymitis, decrease of sperm motility, or hydrosalpinx, etc.); agents for regulating the differentiation and/or function of germ cells; or agents for regulating the differentiation and/or proliferation of blood cells.

For example, when a patient is suffering from insufficient or abnormal signal transduction resulted from decrease or deficiency of the polypeptide of the invention in his/her body, it is possible to restore sufficient or normal function of the polypeptide of the invention by (1) administering the DNA of the invention to the patient and thereby allowing the polypeptide of the invention to be expressed in the body; (2) introducing the DNA of the invention into cells to thereby allow the expression of the polypeptide of the invention, and then transplanting the cells into the patient; or (3) administering the polypeptide of the invention to the patient.

When the DNA of the invention is used as the above-mentioned therapeutic and/or prophylactic, the DNA per se or the DNA inserted into an appropriate vector such as a retrovirus vector, adenovirus vector, adeno-associated virus vector, etc. may be administered to human or other warm-blooded animals using conventional means. The DNA of the invention may be administered as it is or after formulation with physiologically acceptable carriers such as adjuvants to promote uptake, by means of a gene gun or a catheter such as hydrogel catheter.

When the polypeptide of the invention is used as the above-described therapeutic and/or prophylactic, at least 90%, preferably 95% or more, more preferably 98% or more, still preferably 99% or more purified polypeptide of the invention is used.

The polypeptide of the invention may be used, for example, orally in the form of tablets (sugar-coated, if necessary), capsules, elixirs, microcapsules or the like; or parenterally in the form of injections such as aseptic solutions or suspensions in water or other pharmaceutically acceptable liquids. These preparations may be produced, for example, by mixing the polypeptide of the invention with physiologically acceptable carriers, flavoring agents, excipients, vehicles, antiseptics, stabilizers, binders, etc. in unit dosage forms required for preparing generally approved pharmaceutical preparations. The amounts of active ingredients in these formulations are decided so that an appropriate dose within the specified range can be obtained.

Examples of additives which may be mixed in tablets, capsules, etc. include binders such as gelatin, corn starch, tragacanth and gum arabic, excipients such as crystalline cellulose, swelling agents such as corn starch, gelatin and alginic acid, lubricants such as magnesium stearate, sweetening agents such as sucrose, lactose and saccharin, and flavoring agents such as peppermint, akamono oil and cherry. When the unit dosage form is capsule, liquid carrier such as oils and fats may further be included in addition to the above-mentioned materials. Sterile compositions for injection can be formulated according to conventional practices in pharmaceutical manufacturing, e.g., by dissolving or suspending active ingredients, naturally occurring vegetable oils such as sesame oil, coconut oil, etc. in vehicles such as water for injection.

Examples of aqueous liquids for injection include physiological saline and isotonic solutions containing glucose and other auxiliary agents (e.g. D-sorbitol, D-mannitol, sodium chloride, etc.). They may be used in combination with a suitable auxiliary solubilizer such as alcohol (e.g. ethanol, etc.), polyalcohol (e.g. propylene glycol, polyethylene glycol, etc.), nonionic surfactant (e.g. Polysorbate 80™, HCO-50, etc.). Examples of oily liquids for injection include sesame oil, soybean oil, etc. They may be used in combination with an auxiliary solubilizer such as benzyl benzoate, benzyl alcohol, etc. In addition, buffers (e.g. phosphate buffer, sodium acetate buffer, etc.), analgesic agents (e.g. benzalkonium chloride, procaine hydrochloride, etc.), stabilizers (e.g. human serum albumin, polyethylene glycol, etc.), preservatives (e.g. benzyl alcohol, phenol, etc.), antioxidants, etc. may also be admixed therewith. Usually, the prepared injections are filled in appropriate ampoules.

Vectors into which the DNA of the invention has been introduced may also be formulated as described above and usually used parenterally.

Since the thus obtained preparations are safe and of low toxicity, they can be administered to mammals (e.g., human, rat, mouse, guinea pig, rabbit, sheep, pig, bovine, horse, cat, dog, monkey, etc.).

Dose levels of the polypeptide of the invention may vary depending upon the target disease, the patient to be treated, administration route, and so on. When the polypeptide of the invention is administered orally for treating cancer, generally the polypeptide of the invention is administered to adult patients (60 kg in body weight) at a dose of about 1-1000 mg/day, preferably about 10-500 mg/day, more preferably about 10-200 mg/day. With respect to parenteral administration, when the polypeptide of the invention is administered to adult patients (60 kg in body weight) in the form of an injection for treating cancer, it is convenient to inject the polypeptide of the invention into the affected part of the body at a dose of about 1-1000 mg/day, preferably about 1-200 mg/day, and more preferably about 10-100 mg/day, though the dose per administration may vary depending on the patient to be treated, the target disease, etc. For other animals, corresponding doses may be administered after conversion of the above-mentioned values per 60 kg based on actual body weights.

(2) Screening for Candidate Compounds for Medicine to Treat Diseases

Since the polypeptide of the invention exists as a humoral factor in living bodies (especially in thymus, spleen, liver, pancreas, lymph nodes, testis and blood), compounds, or salts thereof, that promote the function of the polypeptide of the invention may be used as medicines, for example, therapeutics and/or prophylactics for various diseases such as immunological diseases, infections (e.g. influenza, tuberculosis, gonococcal infection, viral hepatitis, vancomycin-resistant infection, methicillin-resistant infection, herpes, chlamydia, or sexually transmitted diseases (STD)), gastrointestinal diseases, circulatory organ diseases, endocrine diseases (e.g. abnormality in secondary sex characters, or feminization syndrome) or sterility (e.g. disroders of sperm production, epididymitis, decrease of sperm motility, or hydrosalpinx, etc.); agents for regulating the differentiation and/or function of germ cells; or agents for regulating the differentiation and/or proliferation of blood cells.

On the other hand, compounds, or salts thereof, that inhibit the function of the polypeptide of the invention may be used as medicines such as therapeutics and/or prophylactics for diseases resulted from excessive production of the polypeptide of the invention.

Thus, the polypeptide of the invention is useful as a reagent for screening for compounds, or salts thereof, that promote or inhibit the function of the polypeptide of the invention.

Accordingly, the present invention provides:

(1) a method for screening for compounds, or salts thereof, that promote the function of the polypeptide of the invention or salts thereof (hereinafter, sometimes referred to as the “promoter(s)”), or compounds, or salts thereof, that inhibit the function of the polypeptide of the invention or salts thereof (hereinafter, sometimes referred to as the “inhibitor(s)”).

The screening kit of the invention contains the polypeptide of the invention or a salt thereof.

Compounds or salts thereof obtainable by using the screening method or screening kit of the invention are compounds that are selected from peptides, proteins, non-peptidic compounds, synthetic compounds, fermentation products, cell extracts, plant extracts, animal tissue extracts, plasma and so forth and yet promote or inhibit the function of the polypeptide of the invention.

As salts of such compounds, the same salts as described earlier on the salts of the polypeptide of the invention may be used.

When a compound obtainable by using the screening method or screening kit of the invention is used as the above-described therapeutic and/or prophylactic, the compound may be used by conventional means. For example, the compound may be formulated into tablets, capsules, elixirs, microcapsules, aseptic solutions, suspensions, etc. in the same manner as described for the medicine comprising the polypeptide of the invention.

Since the thus obtained preparations are safe and of low toxicity, they can be administered to mammals (e.g. human, mouse, rat, rabbit, sheep, pig, bovine, horse, cat, dog, monkey, etc.).

Dose levels of these compounds or salts thereof may vary depending upon the target disease, the patient to be treated, administration route, and so on. For example, when a compound that promotes the function of the polypeptide of the invention is administered orally for treating cancer, generally the compound is administered to adult patients (60 kg in body weight) at a dose of about 0.1-100 mg/day, preferably about 1.0-50 mg/day, more preferably about 1.0-20 mg/day. With respect to parenteral administration, when a compound that promotes the function of the polypeptide of the invention is administered to adult patients (60 kg in body weight) in the form of an injection for treating cancer, it is convenient to intravenously inject the compound at a dose of about 0.01-30 mg/day, preferably about 0.1-20 mg/day, and more preferably about 0.1-10 mg/day, though the dose per administration may vary depending on the patient to be treated, the target disease, etc. For other animals, corresponding doses may be administered after conversion of the above-mentioned values per 60 kg based on actual body weights.

On the other hand, when a compound that inhibits the function of the polypeptide of the invention is administered orally, generally the compound is administered to adult patients (60 kg in body weight) at a dose of about 0.1-100 mg/day, preferably about 1.0-50 mg/day, more preferably about 1.0-20 mg/day. With respect to parenteral administration, a compound that inhibits the function of the polypeptide of the invention is administered to adult patients (60 kg in body weight) in the form of an injection, it is convenient to intravenously inject the compound at a dose of about 0.01-30 mg/day, preferably about 0.1-20 mg/day, and more preferably about 0.1-10 mg/day, though the dose per administration may vary depending on the patient to be treated, the target disease, etc. For other animals, corresponding doses may be administered after conversion of the above-mentioned values per 60 kg based on actual body weights.

(3) Quantitative Determination of the Polypeptide of the Invention or Salts Thereof

Since the antibody of the invention can specifically recognize the polypeptide of the invention, the antibody may be used for quantitative determination of the polypeptide of the invention contained in a sample solution. In particular, the antibody may be used in quantitative determination by sandwich immunoassay.

The present invention provides:

(i) a method of quantitative determination of the polypeptide of the invention in a sample solution, comprising reacting the antibody of the invention with the sample solution and the polypeptide of the invention labeled, competitively and determining the ratio of the labeled polypeptide of the invention bound to the antibody; and

(ii) a method of quantitative determination of the polypeptide of the invention in a sample solution, comprising reacting the sample solution with the antibody of the invention insolubilized on a carrier and another antibody of the invention labeled, simultaneously or in succession and determining the activity of the label on the insolubilized carrier.

Further, the monoclonal antibody of the invention may be used to quantitatively determine the polypeptide of the invention or may be used for detection of the polypeptide by tissue staining. For these purposes, either antibody molecules per se or the F(ab′)₂, Fab′ or Fab fragment thereof may be used.

Methods of quantitative determination of the polypeptide of the invention using the antibody of the invention are not particularly limited. Any measuring method may be used in which the amount of antibody, antigen or antibody-antigen complex corresponding to the amount of the antigen in a sample solution (e.g. the amount of the polypeptide of the invention) is detected by chemical of physical means, and then calculated from a standard curve prepared with a standard solution containing a known amount of the antigen. For example, nephrometry, competitive method, immunometric method and sandwich method may be used suitably and, in terms of sensitivity and specificity, the sandwich assay described later is particularly preferred.

Examples of labeling agents used in measuring methods utilizing labeling substances include radioisotopes, enzymes, fluorescent substances, and luminescent substances. Examples of radioisotopes include [¹²⁵I], [¹³¹I], [³H] and [¹⁴C]. Preferred examples of enzymes are those which are stable and with high specific activity, e.g., -galactosidase, -glucosidase, alkali phosphatase, peroxidase and malate dehydrogenase. Examples of fluorescent substances include fluorescamine and fluorescein isothiocyanate. Examples of luminescent substances include luminol, luminol derivatives, luciferin, and lucigenin. Further, a biotin-avidin system may also be used for binding an antibody or antigen with a labeling agent.

Insolubilization of antigens or antibodies may be performed by physical adsorption or by chemical binding usually used for insolubilizing or immobilizing polypeptides or enzymes. Examples of carriers useful for this purpose include insoluble polysaccharides such as agarose, dextran and cellulose; synthetic resins such as polystyrene, polyacrylamide and silicone; and glass.

In the sandwich assay, a sample solution is reacted with an insolubilized monoclonal antibody of the invention (primary reaction); then, another monoclonal antibody of the invention that is labeled is reacted therewith (secondary reaction); and the activity of the labeling agent on the insolubilized carrier is measured to thereby quantitatively determine the amount of the polypeptide of the invention in the sample solution. The primary reaction and the secondary reaction may be conducted in a reverse order, or they may be conducted simultaneously or with an interval. The type of the labeling agent and the method of insolubilization may be the same as those described herein earlier. In immunoassays using the sandwich technique, the antibody insolubilized on a solid phase or the antibody labeled is not necessarily a single antibody; a mixture of two or more antibodies may be used for the purposes of enhancing the sensitivity of measurement, etc.

In the method of measuring the polypeptide of the invention by the sandwich assay of the invention, the monoclonal antibodies of the invention used in the primary and the secondary reactions are preferably those antibodies wherein their sites binding to the polypeptide of the invention are different from each other. For example, if the antibody used in the secondary reaction recognizes the C-terminal region of the polypeptide of the invention, an antibody that recognizes a site other than the C-terminal region, e.g. an N-terminal region, is preferably used in the primary reaction.

The monoclonal antibody of the invention may be used in a measuring system other than the sandwich assay, such as competitive methods, immunometric methods and naphrometry.

In competitive methods, an antigen in a sample solution and a labeled antigen are reacted competitively with an antibody; then, unreacted labeled antigen (F) and labeled antigen bound to the antibody (B) are separated (i.e. B/F separation); and the amount of the label of B or F to thereby quantitatively determine the amount of the antigen in the sample solution. With respect to this reaction method, there are a liquid phase method in which a soluble antibody is used and the B/F separation is conducted with polyethylene glycol and a second antibody to the above-mentioned antibody; and a solid phase method in which a solidified antibody is used as the first antibody or a soluble antibody is used as the first antibody while a solidified antibody is used as the second antibody.

In immunometric methods, an antigen in a sample solution and a solidified antigen are reacted competitively with a specific amount of a labeled antibody, followed by separation of the solid phase from the liquid phase; or an antigen in a sample solution is reacted with an excessive amount of a labeled antibody, and then a solidified antigen is added to bind unreacted labeled antibody to the solid phase, followed by separation of the solid phase from the liquid phase. Subsequently, the amount of label in one of the phases is measured to determine the amount of the antigen in the sample solution.

In nephrometry, the amount of insoluble precipitate generated as a result of antigen-antibody reaction in a gel or solution is measured. Even when the amount of the antigen in a sample solution is small and thus only a small amount of such precipitate is obtained, laser nephrometry utilizing the scattering of laser can be used suitably.

In applying each of those immunological measuring methods to the measuring method of the present invention, no special conditions or operations are required. A measuring system for the polypeptide of the present invention may be constructed using the conventional conditions and operational procedures in the relevant measuring method while taking into account usual technical consideration of those skilled in the art. For details of these commonly used technical means, a variety of reviews, reference books, etc. may be referred to.

For example, Hiroshi Irie (ed.): “Radioimmunoassay” (Kodansha, 1974); Hiroshi Irie (ed.): “Radioimmunoassay; Second Series” (Kodansha, 1979); Eiji Ishikawa et al. (ed.): “Enzyme Immunoassay” (Igaku Shoin, Japan, 1978); Eiji Ishikawa et al. (ed.): “Enzyme Immunoassay” (Second Edition) (Igaku Shoin, 1982); Eiji Ishikawa et al. (ed.): “Enzyme Immunoassay” (Third Edition) (Igaku Shoin, 1987); “Methods in Enzymology”, Vol. 70 (Immunochemical Techniques (Part A)); ibid., Vol. 73 (Immunochemical Techniques (Part B)); ibid., Vol. 74 (Immunochemical Techniques (Part C)); ibid., Vol. 84 (Immunochemical Techniques (Part D: Selected Immunoassays)); ibid., Vol. 92 (Immunochemical Techniques (Part E: Monoclonal Antibodies and General Immunoassay Methods)); ibid., Vol. 121 (Immunochemical Techniques (Part I: Hybridoma Technology and Monoclonal Antibodies)) (Academic Press) and the like may be referred to.

By using the antibody of the invention as described above, the polypeptide of the invention can be quantitatively determined with high sensitivity.

Further, by quantitatively determining the concentration of the polypeptide of the invention using the antibody of the invention, it is possible to diagnose that a subject has a disease(s) such as cancers, immunological diseases, infections, gastrointestinal diseases, circulatory organ diseases or endocrine diseases, or that a subject is very likely to develop such a disease in the future, (1) when an increase or decrease is detected in the concentration of the polypeptide of the invention in the subject.

Further, the antibody of the invention may be used for detecting the polypeptide of the invention present in body fluids, tissues or other samples. The antibody of the invention may also be used in the preparation of antibody columns for use in the purification of the polypeptide of the invention; in the detection of the polypeptide of the invention in individual fractions generated in the course of purification; and in the analysis of the behavior of the polypeptide of the invention in test cells.

(4) Gene Diagnostics

The DNA of the invention can, when used as a probe for example, can detect abnormalities (gene abnormalities) in DNA or mRNA encoding the polypeptide of the invention in mammals (e.g. human, rat, mouse, guinea pig, rabbit, sheep, pig, bovine, horse, cat, dog, monkey, etc.). Thus, the DNA of the invention is useful as a gene diagnostic for diagnosing, e.g., damage, mutations or reduced expression of the above DNA or mRNA, or increase or excessive expression of the above DNA or mRNA.

Gene diagnosis using the DNA of the invention may be performed by known methods such as Northern hybridization or PCR-SSCP method (Genomics, Vol. 5, 874-879 (1989); Proceedings of the National Academy of Sciences of the USA, 86: 2766-2770 (1989)).

When a decrease in expression is detected by Northern hybridization or when a mutation(s) is/are detected in the DNA by PCR-SSCP method, for example, it is possible to diagnose that the relevant subject is very likely to have a disease(s) such as cancers, immunological diseases, infections, gastrointestinal diseases, circulatory organ diseases, endocrine diseases or sterility.

(5) Medicines Containing Antisense DNA

Antisense DNA that complementarily binds to the DNA of the invention and thus inhibits the expression of the DNA of the invention can inhibit the function of the polypeptide or the DNA of the invention in vivo. Therefore, the antisense DNA may be used as therapeutics and/or prophylactics for diseases resulted from excessive expression of the polypeptide of the invention.

The above-mentioned antisense DNA may be used as the above-mentioned therapeutics and/or prophylactics in the same manner as the various therapeutics and/or prophylactics containing the DNA of the invention described earlier.

For example, the antisense DNA per se or the antisense DNA inserted into an appropriate vector such as a retrovirus vector, adenovirus vector, adeno-associated virus vector, etc. may be administered using conventional means. The antisense DNA may be administered as it is or after formulation with physiologically acceptable carriers such as adjuvants to promote uptake, by means of a gene gun or a catheter such as hydrogel catheter.

Further, the antisense DNA may be used as an oligonucleotide probe for diagnostic purposes to examine the presence or state of expression of the DNA of the invention in tissues or cells.

(6) Medicines Containing the Antibody of the Invention

The antibody of the invention that has an effect of neutralizing the activity of the polypeptide of the invention may be used as therapeutics and/or prophylactics for diseases resulted from excessive expression of the polypeptide of the invention.

The above-mentioned therapeutics and/or prophylactics comprising the antibody of the invention may be administered orally or parenterally to mammals (e.g. human, rat, rabbit, sheep, pig, bovine, cat, dog, monkey, etc.) in the forms of liquid preparations without any processing or in appropriate forms of pharmaceutical compositions. Dose levels may vary depending upon the patient to be treated, the target disease, symptoms, administration route, and so on. However, it is convenient to inject the antibody of the invention intravenously at a dose of about 0.01-20 mg/kg body weight, preferably about 0.1-10 mg/kg body weight, more preferably about 0.1-5 mg/kg body weight per administration about one to five times a day, preferably about one to three times a day. In other parenteral administration and oral administration, similar dose levels may be used. If symptoms are particularly heavy, the dose may be increased accordingly.

The antibody of the invention may be administered per se or in the forms of appropriate pharmaceutical compositions. The pharmaceutical compositions for the above administration comprise the antibody or salt thereof, pharmacologically acceptable carriers, and diluents or excipients. Such compositions are provided in forms appropriate for oral or parenteral administration.

For example, compositions for oral administration include solid or liquid preparations such as tablets (including sugar-coated tablets and film-coated tablets), pills, granules, dispersants, capsules (including soft capsules), syrups, emulsions and suspensions. These compositions are -prepared according to conventional methods and contain carriers, diluents or excipients conventionally used in the field of medicine manufacture. For example, lactose, starch, sucrose, magnesium stearate and the like are used as carriers or excipients for tablets.

Compositions for parenteral administration include, for example, injections and suppositories. Injections include intravenous injections, subcutaneous injections, intradermal injections, muscle injections, instilment injections, etc. Such injections may be prepared by dissolving, suspending or emulsifying the above antibody or salt thereof in an aseptic, aqueous or oily liquid. Examples of aqueous liquids for injection include physiological saline and isotonic solutions containing glucose and other auxiliary agents. They may be used in combination with a suitable auxiliary solubilizer such as alcohol (e.g. ethanol), polyalcohol (e.g. propylene glycol, polyethylene glycol), nonionic surfactant [e.g. Polysorbate 80™, HCO-50 (polyoxyethylene (50 mol) adduct of hydrogenated castor oil)], etc.). Examples of oily liquids for injection include sesame oil and soybean oil. They may be used in combination with an auxiliary solubilizer such as benzyl benzoate, benzyl alcohol, etc. Usually, the prepared injections are filled in appropriate ampoules. Suppositories for administration into rectum may be prepared by mixing the antibody or a salt thereof with a conventional suppository base.

It is convenient to formulate the above-described pharmaceutical compositions for oral or parenteral administration into unit dosage forms that would give an appropriate dose of the active ingredient. Examples of such unit dosage forms include tablets, pills, capsules, injections (ampoules), and suppositories. Usually, each unit of these dosage forms contains preferably about 5-500 mg of the above-described antibody. In particular, each unit contains preferably about 5-100 mg in injections, and each unit in other dosage forms contains preferably about 10-250 mg.

The above-described pharmaceutical compositions may contain other active ingredients as long as they do not produce undesirable interaction with the above-described antibody.

(7) DNA-Transferred Animals

The present invention further provides non-human mammals harboring a foreign DNA coding for the polypeptide of the invention (hereinafter referred to briefly as the “foreign DNA of the invention”) or a mutant thereof (sometimes referred to briefly as the “foreign mutant DNA of the invention”).

Thus, the present invention provides:

(1) A non-human mammal harboring the foreign DNA of the invention or a mutant DNA thereof:

(2) The non-human mammal according to (1) which is a rodent:

(3) The non-human mammalian according to (2) wherein the rodent is mouse or rat; and

(4) A recombinant vector containing the foreign DNA of the invention or a mutant DNA thereof and capable of expressing the DNA in a mammal.

The non-human mammal harboring the foreign DNA of the invention or a mutant DNA thereof (hereinafter referred to briefly as the “DNA-transferred animal of the invention”) can be constructed by transferring the DNA of interest to a germinal cell such as unfertilized egg cells, fertilized egg cells, or sperm cells or primordial cells thereof, preferably in the period of embryogenesis in the ontogenesis of the non-human mammal (more preferably, in the stage of a single cell or a fertilized egg cell and generally at the 8-cell stage or earlier), by the calcium phosphate method, electric pulse method, lipofection method, agglutination method, microinjection method, particle gun method, or DEAE-dextran method. It is also possible to transfer the foreign DNA of the invention of interest into somatic cells, organs in the living body, tissue cells, or the like by such DNA transfer methods to use the resultant cells or tissues in cell culture or tissue culture. Further, by fusing the resultant cells with the above-mentioned germinal cell by known cell fusion methods, it is also possible to create the DNA-transferred animal of the invention.

The non-human mammal used includes bovine, pig, sheep, goat, rabbit, dog, cat, guinea pig, hamster, mouse, rat, and so on. From the viewpoint of construction of diseased animal models, rodents which have comparatively short ontogenesis and life cycles and can be easily bred, particularly mouse (e.g. pure strains such as C57BL/6, DBA2, etc. and hybrid strains such as B6C3F₁, BDF₁, B6D2F₁, BALB/c, ICR, etc.) or rat (e.g. Wistar, SD, etc.), are preferred.

The foreign DNA of the invention is not a DNA of the invention which is inherently possessed by the non-human mammal, but a DNA of the invention that has been once isolated or extracted from a mammal.

Examples of the mutant DNA of the invention include not only the DNAs that have variations (e.g. mutations) in the nucleotide sequence of the original DNA of the invention, for example, upon addition or deletion of nucleotides or substitution with other nucleotides, but also abnormal DNAs.

The term “abnormal DNA” as used herein means any DNA that causes expression of an abnormal polypeptide of the invention. For example, a DNA that allows expression of a polypeptide inhibiting the function of the normal polypeptide of the invention may be used.

The foreign DNA of the invention may be derived from a mammal that is of the same species as that of the host animal or of different species. For transfer of the DNA of the invention to the host animal, it is generally advantageous to use a DNA construct in which the DNA is ligated downstream of a promoter capable of expressing the DNA in animal cells. For example, in transferring the human DNA of the invention, this human DNA of the invention may be ligated downstream of a promoter capable of directing expression of DNAs derived from various animals (e.g. rabbit, dog, cat, guinea pig, hamster, rat, mouse, etc.) harboring the DNA of the invention having high homology to the human DNA to thereby prepare a DNA construct (e.g. vector), which can then be microinjected into fertilized egg cells of a host mammal such as fertilized mouse egg cells. Thus, a DNA-transferred mammal showing high expression of the DNA of the invention can be provided.

Examples of the expression vector for the polypeptide of the invention include plasmids derived from E. coli, plasmids derived from B. subtilis, plasmids derived from yeast, phage and other bacteriophages, retroviruses such as Molony leukemia virus, and animal viruses such as vaccinia virus and vaculovirus. Preferable examples are E. coli-derived plasmids, B. subtilis-derived plasmids and yeast-derived plasmids.

Examples of promoters that regulate the expression of the DNA include (1) promoters for DNAs derived from viruses (e.g. simian virus, cytomegalovirus, Molony leukemia virus, JC virus, papilloma virus, poliovirus, etc.), (2) promoters derived from mammals (e.g. human, rabbit, dog, cat, guinea pig, hamster, rat, mouse, etc.), for exmaple, promoters of albumin, insulin II, uroprakin II, elastase, erythropoietin, endothelin, muscle creatine kinase, glial fibrillary acidic polypeptide, glutathione S-transferase, platelet-derived growth factor, keratin K1, K10, and K14, collagen type I and type II, cyclic AMP-dependent polypeptide kinase I subunit, dystrophin, tartaric acid-resistant alkaline phosphatase, atrial natriuretic factor, endothelial receptor tyrosine kinase (generally abbreviated to Tie2), sodium/potassium-dependent adenosinetriphosphatase (Na, K-ATPase), neurofilament light chain, metallothionein I and IIA, metalloproteinase I tissue inhibitor, MHC Class I antigen (H-2L), H-ras, renin, dopamine -hydroxylase, thyroid peroxidase (TPO), polypeptide chain elongation factor 1 (EF-1), -actin, -and -myosin heavy chain, myosin light chains 1 and 2, myelin basic polypeptide, thyroglobulin, Thy-1, immunoglobulin, H chain variable region (VNP), serum amyloid P component, myoglobin, troponin C, smooth muscle -actin, preproenkephalin A, vasopressin, and so on. Preferable are those promoters which can direct high expression of the DNA in the whole body, e.g. cytomegalovirus promoter, human polypeptide chain elongation factor 1 (EF-1) promoter, and human and chicken -actin promoters.

It is preferable that the vector has a sequence for terminating the transcription of the mRNA of interest (generally called terminator) in the DNA-transferred mammal. For example, sequences derived from viruses or various mammals may be used. Preferably, SV40 terminator derived from simian virus or the like is used.

In addition, for enhancing the expression of the DNA of interest further, it is possible, depending on the specific purpose, to ligate a splicing signal, an enhancer region, a portion of an eucaryotic DNA intron, etc. upstream of the 5′-end of the promoter region, between the promoter region and the translation region, or downstream of the 3′-end of the translation region.

The translation region can be prepared as a DNA construct which can be expressed in a DNA-transferred animal, by conventional recombinant DNA techniques, i.e. by ligating it downstream of the promoter and, if desired, upstream of the transcription termination site.

The transfer of the foreign DNA of the invention at the fertilized egg cell stage insures that the DNA will be ubiquitous in all the germ cells and somatic cells of the host mammal. The presence of the DNA of the invention in the germ cells of the DNA-transferred animal following DNA transfer means that all the germinal cells and somatic cells of all the subsequent generations of the DNA-transferred animal harbor the foreign DNA of the invention. Thus, the progeny of such DNA-transferred animal which inherited the foreign DNA of the invention have the foreign DNA in all of their germ cells and somatic cells.

The non-human mammal harboring the foreign normal DNA of the invention can be verified by mating to retain the foreign DNA stably and then bred as a line harboring that DNA from generation to generation under usual breeding conditions.

The transfer of the foreign DNA of the invention at the fertilized egg cell stage insures that the DNA will be present in excess in all the germ cells and somatic cells of the host mammal. The presence of the foreign DNA of the invention in the germ cells of the DNA-transferred animal following the DNA transfer means that all the germinal cells and somatic cells of all the progeny of the DNA-transferred animal harbor the foreign DNA of the invention in excess. Thus, the progeny of such DNA-transferred animal which inherited the foreign DNA of the invention have the DNA in excess in their germ cells and somatic cells.

By preparing homozygous animals having the transferred DNA in both homologous chromosomes and mating male animals with female animals, it is possible to breed-through generations so that every progeny harbors the DNA in excess.

The non-human mammal harboring the normal DNA of the invention features a high expression of the DNA and may eventually develop a hyperergasia of the polypeptide of the invention through promotion of the function of the endogenous normal DNA. Thus, the animal can be utilized as an animal model of that disease. For example, by using the DNA-transferred animal harboring the normal DNA of the invention, it is possible to study the hyperergasia of the polypeptide of the invention to elucidate the mechanisms of diseases with which the polypeptide of the invention is associated, and explore therapeutic modalities for the diseases.

Furthermore, the mammal to which the foreign normal DNA of the invention has been transferred presents symptoms due to an increase in the free polypeptide of the invention and, therefore, can also be used in the screening of therapeutic drugs for diseases with which the polypeptide of the invention is associated.

On the other hand, the non-human mammal harboring the foreign abnormal DNA of the invention can be verified by mating to retain the DNA stably and then bred as a line harboring the DNA from generation to generation under usual breeding conditions. Moreover, it is possible to incorporate the DNA of interest in the above-mentioned plasmid for use as a starting material. The DNA construct with the promoter can be prepared by conventional recombinant DNA techniques. Transfer of the abnormal DNA of the invention in the fertilized egg cell stage insures that the transferred DNA will be ubiquitous in all the germ cells and somatic cells of the host mammal. The presence of the abnormal DNA of the invention in the germ cells of the DNA-transferred animal after transferring DNA means that all the progeny of this DNA-transferred animal harbor the abnormal DNA of the invention in all of their germinal cells and somatic cells. The progeny of this animal inherited the foreign DNA of the invention harbor the abnormal DNA of the invention in all of their germinal cells and somatic cells. By preparing homozygous male and female animals having the introduced DNA in both homologous chromosomes and mating them, it can be insured that every progeny harbors the DNA from generation to generation.

The non-human mammal harboring the abnormal DNA of the invention features a high expression of the abnormal DNA and, therefore, may eventually develop adiaphoria associated with functional inactivation of the polypeptide of the invention through inhibition of the function of the endogenous normal DNA. Thus, the animal can be utilized as an animal model of that disease. For example, by using the DNA-transferred animal harboring the abnormal DNA of the invention, analysis of the mechanism of this functional inactivation adiaphoria attributable to the polypeptide of the invention and therapeutic modalities for the disease can be explored.

As a specific potential use, the DNA-transferred animal with a high expression of the abnormal DNA of the invention can be used as a model for elucidating the functional inhibition of the normal polypeptide by the abnormal polypeptide of the invention (dominant negative effect) in adiaphoria of functional inactivation type attributable to the polypeptide of the invention.

Moreover, the DNA-transferred mammal harboring the foreign abnormal DNA of the invention develops symptoms due to an increase in the free polypeptide of the invention and, therefore, can be utilized in the screening of therapeutic drugs for adiaphoria attributable to functional inactivation of the polypeptide of the invention.

As other potential uses of the two types of DNA-transferred animals harboring the two kinds of DNAs of the invention, the following may be considered:

(1) Use as a cell source for tissue culture;

(2) Analysis of those genes or polypeptides which are expressed or activated or deactivated specifically by the polypeptide of the invention, by comparing and analyzing the DNA or RNA in tissues of the DNA-transferred animal of the invention with the DNA or RNA of non-DNA-transferred animal (control animal) or by comparing and analyzing the compositions of the polypeptides expressed;

(3) Study of the functions of cells of those tissues which are generally difficult to culture, by using the cells from the tissues containing the DNA as cultured by the standard tissue culture technique;

(4) Screening for drugs capable of enhancing the cell functions by using the cells described in (3); and

(5) Isolation and purification of the mutant polypeptide of the invention and construction of antibodies thereto.

Furthermore, by using the DNA-transferred animal of the invention, clinical symptoms of diseases associated with the polypeptide of the invention, inclusive of above-described adiaphoria associated with functional inactivation of the polypeptide of the invention, can be investigated. In addition, more detailed pathological findings can be obtained in various organs of this model of diseases associated with the polypeptide of the invention, thus contributing to the development of new therapies as well as the study and treatment of secondary diseases arising from such diseases.

Moreover, by removing various organs from the DNA-transferred animal of the invention, mincing them and digesting them with a proteolytic enzyme such as trypsin, free single cells harboring the transferred DNA can be recovered. These cells can be cultured for establishment of a cell line. Furthermore, characterization of cells producing the polypeptide of the invention can be made and their relationship with apoptosis, differentiation, or proliferation, the mechanism of signal transduction in them, and abnormalities involved can be explored to thereby generate information useful for further elucidation of the polypeptide of the invention and its effects.

Moreover, for the development of therapeutic drugs for diseases associated with the polypeptide of the invention, such as adiaphoria resulted from functional inactivation of the polypeptide of the invention by using the DNA-transferred animal of the invention, an effective and rapid screening technology for such therapeutic drugs can be established by using the test and assay methods described hereinbefore. In addition, by using the above DNA-transferred animal or the foreign DNA expression vector of the invention, gene therapies for diseases associated with the polypeptide of the invention can be explored and developed.

(8) Knockout Animals

The invention further provides non-human mammalian embryonic stem cells wherein the DNA of the invention is inactivated, and non-human mammals deficient in expression of the DNA of the invention wherein the DNA of the invention is deactivated.

The invention, therefore, provides:

(1) A non-human mammalian embryonic stem cell wherein the DNA of the invention is inactivated;

(2) The embryonic stem cell according to in (1) wherein the DNA is inactivated by introduction of a reporter gene (e.g. E. coli-derived -galactosidase gene);

(3) The embryonic stem cell according to (1) which is neomycin-resistant;

(4) The embryonic stem cell according to (1) wherein the non-human mammal is a rodent;

(5) The embryonic stem cell according to (4) wherein the rodent is mouse;

(6) A non-human mammal deficient in expression of the DNA of the invention, wherein the DNA is inactivated;

(7) The non-human mammal according to (6) wherein the DNA is inactivated by introduction of a reporter gene (e.g. E. coli-derived -galactosidase gene) and the reporter gene can be expressed under the control of the promoter for the DNA of the invention;

(8) The non-human mammal according to (6) wherein the non-human mammal is a rodent;

(9) The non-human mammal according to (8) wherein the rodent is mouse; and

(10) A method for screening for compounds, or salts thereof, that enhance or inhibit the promoter activity for the DNA of the invention, which comprises administering a test compound to the non-human mammal according to (7) and detecting expression of the reporter gene.

The term “non-human mammalian embryonic stem cell wherein the DNA of the invention is inactivated” means the embryonic stem cell (hereinafter referred to briefly as ES cell) of a non-human mammal in which the DNA has been deprived of the capacity to express the polypeptide of the invention (hereinafter referred to sometimes as the “knockout DNA of the invention”) through introduction of an artificial mutation to the DNA of the invention possessed by the non-human mammal to thereby inhibit expression of the DNA of the invention or through substantial deprivation of the activity of the polypeptide of the invention encoded by the DNA.

As the non-human mammals, the same animals as mentioned hereinbefore may be used.

Examples of the method for introducing an artificial mutation to the DNA of the invention are a deletion of some or all of the DNA sequence, or an insertion of a different DNA, or substitution with a different DNA by the genetic engineering technology. The knockout DNA of the invention may be created by such a mutation that would shift the reading frame or destroy the function of the promoter or exon.

The non-human mammalian embryonic stem cell wherein the DNA of the invention is inactivated (hereinafter referred to as the “ES cell wherein the DNA of the invention is inactivated or the “knockout ES cell of the invention”) can be prepared by, for example, procedures which comprise isolating the DNA of the invention from a non-human mammal of interest, inserting a drug-resistance gene, typically neomycin-resistance gene or hygromycin-resistance gene, or a reporter gene such as lacZ (-galactosidase gene) or cat (chloramphenicol acetyltransferase gene) into its exon region to disrupt the function of the exon or inserting a DNA sequence for terminating gene transcription (e.g. poly A addition signal) in an intron region between exons to thereby inhibit synthesis of a complete mRNA, introducing the thus-constructed DNA strand having a DNA sequence designed to eventually disrupt the gene (hereinafter, referred to briefly as the “targeting vector”) into the chromosomes of the host animal by homologous recombination, subjecting the resulting ES cell to Southern hybridization analysis using a DNA sequence located on the DNA of the invention or in its vicinity as a probe or a PCR procedure using a DNA sequence located on the targeting vector and a DNA sequence in the vicinity but not including the DNA of the invention used in the construction of the targeting vector as primers, and selecting the knockout ES cell of the invention.

The original ES cell used for inactivation of the DNA of the invention by the homologous recombination technique or the like may be an already established cell line such as those mentioned hereinbefore or a new cell line established de novo by the known method of Evans and Kaufma. Taking mouse ES cells as an example, ES cells of the 129 line are generally employed but the immunological background of this line is not clear. Therefore, the cell line established by using BDF₁ mice created by the hybridization of C57BL/6 mice and C57BL/6 mice, both yielding few eggs, with DBA/2 mice (BDF₁=F₁ of C57BL/6 and DBA/2) for preparing pure-line ES cells with an immunologically defined genetic background can be used with advantage. In addition to the advantage of high egg output and sturdiness of the egg, BDF, mice have the background of C57BL/6 mice so that in the construction of a disease model with ES cells obtained, the genetic background of the model mice can be converted to that of C57BL/6 mice by back-crossing with C57BL/6.

Moreover, in establishing an ES cell line, it is common practice to use blastocytes 3.5 days following fertilization but, aside from them, a large number of early embryos can be prepared with high efficiency by harvesting the embryos at the 8-cell stage and culturing them into blastocytes.

Furthermore, while ES cells from both male and female animals can be used, generally ES cells of male animals are more convenient for the construction of reproduction line chimeras. Moreover, for the purpose of reducing the burden of the complicated cultural procedure, it is preferable to carry out sexing as early as possible.

As a typical method for sexing ES cells, there can be mentioned the method in which the gene in the sex determination region on the Y chromosome is amplified and detected by PCR. Whereas the conventional karyotype analysis requires about 10⁶ cells, the above method requires only about one colony equivalent of ES cells (about 50 cells). Therefore, the primary selection of ES cells in an early stage can be made by this sexing method. Since male cells can thus be selected in the early stage, the trouble in the initial stage of culture can be drastically reduced.

Moreover, the secondary selection can be carried out by G-banding for the number of chromosomes. The number of chromosomes in the resulting ES cell is preferably 100% of the normal number but this goal may not be reached due to the physical and other factors involved in the establishment of the line. In such cases, it is preferable to knockout the gene of the ES cell and re-clone it into the normal cell (taking a mouse as an example, the cell in which the number of chromosomes is 2n=40).

The embryonic stem cell line thus established is generally very satisfactory in proliferation characteristic but since it is liable to lose its ontogenic ability, it must be subcultured with sufficient care. For example, this cell line should be cultured on suitable feeder cells such as STO fibroblasts in the presence of LIF (1-10000 U/ml) in a carbon dioxide incubator (preferably 5% CO₂-95% air or 5% oxygen-5% CO₂-90% air) at about 37° C. and, in subculture, it should be treated with trypsin/EDTA solution (generally about 0.001-0.5% trypsin/about 0.1-5 mM EDTA, preferably about 0.1% trypsin/about 1 mM EDTA) to provide single cells and seed them on freshly prepared feeder cells. While such subculture is generally performed every 1-3 days, it is good practice to observe the cells on each occasion and, whenever morphologically abnormal cells are discovered, discard the culture.

ES cells can be allowed to differentiate into various types of cells, such as head long muscle cells, visceral muscle cells, heart muscle cells, etc. by conducting monolayer culture to a high density under suitable conditions or suspension culture until a mass of cells is formed (M. J. Evans & M. H. Kaufman, Nature, 292, 154, 1981; G. R. Martin, Proceedings of National Academy of Science USA, 78, 7634, 1981; T. C. Doetschman et al., Journal of Embryology and Experimental Morphology, 87, 27, 1985), and the cell deficient in expression of the DNA of the invention as obtained by causing the ES cell of the invention to differentiate is useful for the cytobiological in vitro study of the polypeptide of the invention.

The non-human mammal deficient in expression of the DNA of the invention can be differentiated from normal animals by assaying the mRNA in the animals by the known method and comparing the amounts of expression indirectly.

As the non-human mammal used for this purpose, the same animals as mentioned hereinbefore may be used.

With respect to the non-human mammal deficient in expression of the DNA of the invention, the DNA of the invention can be knocked out by introducing the targeting vector constructed as above into, for example, mouse embryonic stem cells or mouse egg cells and thereby allowing the DNA sequence of the targeting vector harboring the inactivated DNA of the invention to undergo homologous recombination with, and accordingly replacing, the DNA of the invention on the mouse embryonic stem cell or egg cell chromosomes.

The cell with the DNA of the invention thus knocked out can be judged by Southern hybridization analysis using a DNA sequence on the DNA of the invention or in its vicinity as a probe or by PCR using a DNA sequence on the targeting vector or a mouse-derived DNA sequence in a region adjacent to but not including the DNA of the invention used in the targeting vector as primers. When a non-human mammalian embryonic stem cell is used, a cell line with the DNA of the invention knocked out by the homologous recombination technique is cloned and injected into the non-human mammalian embryo or blastocyte at a suitable stage of embryogenesis, for example at the 8-cell stage, and the resulting chimera embryo is transplanted in the pseudopregnant uterus of the non-human mammal. The animal thus obtained is a chimera animal constituted by both the cells harboring the normal DNA locus of the invention and the cells harboring the artificially mutated DNA locus of the invention.

When some of the gametes of this chimera animal harbor the mutated DNA locus of the invention, an individual the entire tissues of which are constituted by cells harboring the mutated DNA locus of the invention can be screened from the colony of animals obtained by crossing such a chimera animal with a normal animal, for example by coat color discrimination. The individuals thus selected are usually animals deficient in hetero-expression of the polypeptide of the invention and by mating such individuals deficient in hetero-expression of the polypeptide of the invention with each other, animals deficient in homo-expression of the polypeptide of the invention can be acquired.

When egg cells are used, a transgenic non-human mammal with the targeting vector having been introduced into its chromosomes can be prepared by injecting the DNA solution into the egg cell nucleus by the microinjection technique and selecting animals expressing a mutation of the DNA locus of the invention by homologous recombination.

The individuals with the DNA of the invention knocked out are mated to verify that the animals obtained by mating also have the DNA knocked out and they can be sub-bred under the usual breeding conditions.

Preparation and maintenance of the germ line may also be carried out in accordance with conventional methods. Thus, by mating male and female animals harboring the inactivated DNA, homozygotes having the inactivated DNA in both homologous chromosomes can be obtained. The homozygotes thus obtained are bred under such conditions that, with regard to the dam, the number of homozygotes is plural per normal individual. By mating male and female heterozygotes, homozygotes and heterozygotes both harboring the inactivated DNA can be sub-bread.

The non-human mammalian embryonic stem cell harboring the inactivated DNA of the invention is very useful for the construction of non-human mammals deficient in expression of the DNA of the invention.

Moreover, the mouse deficient in expression of the polypeptide of the invention lacks the various biological activities inducible by the polypeptide of the invention and can, therefore, be of use as an animal model of diseases arising from inactivation of the biological activities of the polypeptide of the invention, thus being useful in the etiological studies of such diseases and development of therapeutic methods.

(8a) Method for Screening for Compounds with Therapeutic/Prophylactic Effect upon Diseases Resulted from Deficiency of or Damage to the DNA of the Invention

Non-human mammals deficient in expression of the DNA of the invention may be used for screening for compounds with a therapeutic and/or prophylactic effect upon diseases resulted from deficiency of or damage to the DNA of the invention (cancers, immunological diseases, infections, gastrointestinal diseases, circulatory organ diseases, endocrine diseases, etc.).

The present invention provides a method for screening for compounds, or salts thereof, having a therapeutic and/or prophylactic effect upon diseases resulted from deficiency of or damage to the DNA of the invention, which is characterized by administering a test compound to a non-human mammal deficient in expression of the DNA of the invention and observing and measuring the changes in the mammal.

As the non-human mammal deficient in expression of the DNA of the invention, the same animals as described earlier may be used.

The test compound may be, for example, a peptide, protein, non-peptidic compound, synthetic compound, fermentation product, cell extract, plant extract, animal tissue extract or plasma. These compounds may be either novel compounds or known compounds.

Specifically, a non-human mammal deficient in expression of the DNA of the invention is treated with a test compound and then compared with a control animal not treated with the compound. Subsequently, the therapeutic and/or prophylactic effect of the test compound may be examined using the changes in individual organs, tissues or disease symptoms in the mammal.

As a method for treating a test animal with a test compound, oral administration, intravenous injection, or the like may be used. The method may be appropriately selected depending on the symptoms of the test animal, the nature of the test compound, and so on. Dose levels of the test compound may be appropriately selected taking into account of the administration method, the nature of the test compound, and so on.

Compounds obtainable by using the screening method of the invention are compounds that are selected from the above-mentioned test compounds, and have a therapeutic and/or prophylactic effect upon diseases resulted from deficiency of or damage to the DNA of the invention (cancers, immunological diseases, infections, gastrointestinal diseases, circulatory organ diseases, endocrine diseases, etc.). Therefore, they may be used as therapeutics and/or prophylactics for those diseases that are safe and of low toxicity. Furthermore, compounds inducible from those compounds obtained by the above screening may also be used in the same manner.

The compound obtained by the above screening may be in a salt form. As salts of the compounds, salts formed with physiologically acceptable acids (e.g. organic or inorganic acids) or bases (e.g. alkali metals) may be used. Especially preferable are physiologically acceptable acid addition salts. Examples of such salts include salts formed with inorganic acids (e.g. hydrochloric acid, phosphoric acid, hydrobromic acid or sulfuric acid) and salts formed with organic acids (e.g. acetic acid, formic acid, propionic acid, fumaric acid, maleic acid, succinic acid, tartaric acid, citric acid, malic acid, oxalic acid, benzoic acid, methanesulfonic acid or benzenesulfonic acid).

Medicines comprising the compound, or a salt thereof, obtained by the screening may be prepared in the same manner as described for medicines comprising the polypeptide of the invention.

Since the thus obtained preparations are safe and of low toxicity, they may be administered to, for example, mammals (such as human, rat, mouse, guinea pig, rabbit, sheep, pig, bovine, horse, cat, dog, monkey, etc.).

Dose levels of the above compound or a salt thereof may vary depending upon the target disease, the patient to be treated, administration route, and so on. When the compound is administered orally for treating cancer, generally the compound is administered to adult patients (60 kg in body weight) at a dose of about 0.1-100 mg/day, preferably about 1.0-50 mg/day, more preferably about 1.0-20 mg/day. With respect to parenteral administration, when the compound is administered to adult patients (60 kg in body weight) in the form of an injection for treating cancer, it is convenient to intravenously inject the compound at a dose of about 0.01-30 mg/day, preferably about 0.1-20 mg/day, and more preferably about 0.1-10 mg/day, though the dose per administration may vary depending on the patient to be treated, the target disease, etc. For other animals, corresponding doses may be administered after conversion of the above-mentioned values per 60 kg based on actual body weights.

(8b) Method for Screening for Compounds that Promote or Inhibit Promoter Activity for the DNA of the Invention

The present invention provides a method for screening for compounds, or salts thereof, that promote or inhibit promoter activity for the DNA of the invention, which is characterized by administering a test compound to a non-human mammal deficient in expression of the DNA of the invention and detecting the expression of a reporter gene.

In the above screening method, there is used a non-human mammal deficient in expression of the DNA of the invention wherein the DNA of the invention is inactivated as a result of introduction of a reporter gene, and this reporter gene is capable of being expressed under the control of the promoter for the DNA of the invention.

As the test compound, the compounds as enumerated above may be used.

As the reporter gene, the genes as enumerated above may be used. Among all, galactosidase gene (lacZ), soluble alkali phosphatase gene or luciferase gene may be preferably used.

In the non-human mammal deficient in expression of the DNA of the invention wherein the DNA of the invention is replaced with a reporter gene, since the reporter gene is present under the control of the promoter for the DNA of the invention, the promoter activity can be detected by tracing the expression of the substance encoded by the reporter gene.

For example, when a part of the DNA region encoding the polypeptide of the invention is replaced with E. coli-derived -galactosidase gene (lacZ), -galactosidase is expressed instead of the polypeptide of the invention in those tissues where originally the polypeptide of the invention has been expressed. Thus, by staining with a reagent such as 5-bromo-4-chloro-3-indolyl- -D-galactopyranoside (X-gal), which is a substrate for -galactosidase, it is possible to observe the state of in vivo expression of the polypeptide of the invention in the mammal simply. Specifically, mice deficient in the polypeptide of the invention or tissue sections thereof may be fixed in glutaraldehyde or the like, washed with phosphate-buffered physiological saline (PBS), and treated with a staining solution containing X-gal at room temperature or around 37 C for about 30 min to 1 hr. Subsequently, the tissue samples are washed with 1 mM EDTA/PBS solution to terminate the -galactosidase reaction, followed by observation of the resultant color development. Alternatively, mRNA encoding lacZ may be detected according to conventional methods.

The compounds or salts thereof obtainable by the above-described screening are compounds that are selected from the above-mentioned test compounds and yet promote or inhibit the promoter activity for the DNA of the invention.

The compound obtained by the above screening may be in a salt form. As salts of the compound, salts formed with physiologically acceptable acids (e.g. inorganic acids) or bases (e.g. organic acids) may be used. Especially preferable are physiologically acceptable acid addition salts. Examples of such salts include salts formed with inorganic acids (e.g. hydrochloric acid, phosphoric acid, hydrobromic acid or sulfuric acid) and salts formed with organic acids (e.g. acetic acid, formic acid, propionic acid, fumaric acid, maleic acid, succinic acid, tartaric acid, citric acid, malic acid, oxalic acid, benzoic acid, methanesulfonic acid or benzenesulfonic acid).

Since compounds, or salts thereof, that promote the promoter activity for the DNA of the invention can promote the expression of the polypeptide of the invention and thereby promote the function thereof, they are useful as therapeutics and/or prophylactics for diseases such as cancers, immunological diseases, infections, digestive tract diseases, circulatory organ diseases or endocrine diseases, the therapeutics and/or prophylactics being safe and of low toxicity.

Further, those compounds inducible from the compounds obtained from the above screening may also be used in the same manner.

Medicines comprising the compound or, a salt thereof, obtained by the screening method may be prepared in the same manner as described for medicines comprising the polypeptide of the invention or a salt thereof.

Since the thus obtained preparations are safe and of low toxicity, they may be administered to, for example, mammals (such as rat, human, mouse, guinea pig, rabbit, sheep, pig, bovine, horse, cat, dog, monkey, etc.).

Dose levels of the above compound or a salt thereof may vary depending upon the target disease, the patient to be treated, administration route, and so on. When a compound that promotes the promoter activity for the DNA of the invention is administered orally for treating cancer, generally the compound is administered to adult patients (60 kg in body weight) at a dose of about 0.1-100 mg/day, preferably about 1.0-50 mg/day, more preferably about 1.0-20 mg/day. With respect to parenteral administration, when a compound that promotes the promoter activity for the DNA of the invention is administered to adult patients (60 kg in body weight) in the form of an injection for treating cancer, it is convenient to intravenously inject the compound at a dose of about 0.01-30 mg/day, preferably about 0.1-20 mg/day, and more preferably about 0.1-10 mg/day, though the dose per administration may vary depending on the patient to be treated, the target disease, etc. For other animals, corresponding doses may be administered after conversion of the above-mentioned values per 60 kg based on actual body weights.

On the other hand, when a compound that inhibits the promoter activity for the DNA of the invention is administered orally, generally the compound is administered to adult cancer patients (60 kg in body weight) at a dose of about 0.1-100 mg/day, preferably about 1.0-50 mg/day, more preferably about 1.0-20 mg/day. With respect to parenteral administration, when a compound that inhibits the promoter activity for the DNA of the invention is administered to adult cancer patients (60 kg in body weight) in the form of an injection, it is convenient to intravenously inject the compound at a dose of about 0.01-30 mg/day, preferably about 0.1-20 mg/day, and more preferably about 0.1-10 mg/day, though the dose per administration may vary depending on the patient to be treated, the target disease, etc. For other animals, corresponding doses may be administered after conversion of the above-mentioned values per 60 kg based on actual body weights.

Thus, the non-human mammal deficient in expression of the DNA of the invention is extremely useful in screening for compounds, or salts thereof, that promote or inhibit the promoter activity to the DNA of the invention, and may contribute greatly to the elucidation of causes of various diseases resulted from deficiency in expression of the DNA of the invention or to the development of prophylactics and/or therapeutics for such diseases.

Further, since the gene encoding the polypeptide of the invention is expressed specifically in such tissues as thymus, spleen, liver, pancreas, lymph nodes, or testis in human, the promoter sequence of the gene is convenient as a promoter for driving high expression of a protein of interest (any useful gene product, etc.) in those tissues of non-human, warm-blooded animals. As the non-human, warm-blooded animal, the warm-blooded animals as enumerated earlier may be used.

The present invention provides a method for allowing predominant expression of a protein of interest (any useful gene product, etc.) in tissues of a non-human, warm-blooded animal such as thymus, spleen, liver, pancreas, lymph nodes or testis, comprising ligating a DNA encoding the protein of interest (any useful gene product, etc.) downstream of the promoter region (i.e. 3′ to the promoter) of a gene encoding a polypeptide comprising the amino acid sequence as shown in SEQ ID NO: 2 and transferring the resultant DNA construct to the non-human animal.

Examples of the protein of interest (any useful gene product, etc.) include cytokines (e.g. interleukin, interferon, chemokine, hematopoietic factor, etc.), growth factors [e.g. EGF (epidermal growth factor) or substances having substantially the same activity such as EGF, halegrin (HER2 ligand), etc.], insulin or substances having substantially the same activity [e.g. insulin, IGF (insulin-like growth factor)-1, IGF-2, etc.], FGF (fibroblast growth factor) or substances having substantially the same activity [e.g. aFGF, bFGF, KGF (keratinocyte growth factor), HGF (hepatocyte growth factor), FGF-10, etc.], other cell proliferation factors [e.g. CSF (colony stimulating factor), EPO (erythropoietin), IL-2 (interleukin-2), NGF (nerve growth factor), PDGF (platelet-derived growth factor), TGF (transforming growth factor), etc.], hormones [e.g. lutenizing hormone-releasing hormone (LH-RH), growth hormone, growth hormone-releasing hormone (GH-RH), prolactin, melanocyte-stimulating hormone, thyroid hormone-releasing hormone, thyrotropic hormone, lutenizing hormone, progesterone, follicle-stimulating hormone, gastrin, motilin, somatostatin, selectin, glucagon, PACAP, VIP, etc., digestive enzymes (e.g. amylase, pepsinogen, lipase, etc.), antibodies to pathogens (e.g. antibodies to pathogenic bacteria such as Salmonella, antibodies to pathogenic viruses such as Influenza, antibodies to parasites such as Echinococcus, etc.) and antimicrobial polypeptides (e.g. cecropin, histatin, indolicidin, protegrin, difencin, lysozyme, etc.).

Of the above-mentioned proteins of interest,

(i) by allowing a cytokine to be expressed specifically in the thymus and the spleen, it is possible to achieve, for example, enhancement or regulation of the immunological activity of non-human, warm-blooded animals; and

(ii) by allowing a growth factor to be expressed specifically in the liver, it is possible to achieve, for example, promotion of the growth of non-human, warm-blooded animals.

Hereinbelow, the method for allowing expression of a protein of interest (any useful gene product, etc.) specifically in thymus, spleen, liver, pancreas, lymph nodes, testis, etc. of a non-human, warm-blooded animal, comprising ligating a DNA or RNA encoding the protein of interest downstream of the promoter region (i.e. 3′ to the promoter) of a gene encoding a polypeptide comprising the amino acid sequence as shown in SEQ ID NO: 2 and transferring the resultant DNA construct to the non-human, warm-blooded animal, will be described more specifically.

First, the promoter of the gene encoding a polypeptide comprising the amino acid sequence as shown in SEQ ID NO: 2 may be obtained by conventional methods such as colony hybridization, plaque hybridization, PCR, etc. (e.g., methods described in Molecular Cloning, 2nd Ed., Sambrook et al., Cold Spring Harbor Lab. Press, 1989). The identification of the region with promoter activity may be performed by known methods such as reporter assay (e.g., methods described in Analytical Biochemistry, Vol. 188, p. 245 (1990)).

Subsequently, the ligation of a protein of interest (any useful gene product, etc.) downstream of the thus obtained promoter (i.e. 3′ to the promoter) may be performed by known methods for constructing plasmids with T4DNA ligase (e.g., methods described in Molecular Cloning, 2nd Ed., Sambrook et al., Cold Spring Harbor Lab. Press, 1989).

For transferring the resultant construct in which a DNA encoding the protein of interest is ligated downstream of (i.e. 3′ to) the promoter, a method using electroporation, a method using a gene gun, a method using a retrovirus vector (e.g. the method described in Blood Cells, Vol. 17, p. 407 (1991)), a method using an adenovirus vector (e.g. the method described in Pathology, Vol. 30, p. 335 (1998)) or the like may be employed.

In the specification and drawings of the present application, the abbreviations used for bases (nucleotides), amino acids and so forth are those recommended by the IUPAC-IUB Commission on Biochemical Nomenclature or those conventionally used in the art. Examples of such abbreviations are given below. Amino acids that may have optical isomers are intended to represent their L-isomer unless otherwise specified.

DNA: Deoxyribonucleic acid

cDNA: Complementary deoxyribonucleic acid

A: Adenine

T: Thymine

G: Guanine

C: Cytosine

RNA: Ribonucleic acid

mRNA: Messenger ribonucleic acid

DATP: Deoxyadenosine triphosphate

dTTP: Deoxythymidine triphosphate

dGTP: Deoxyguanosine triphosphate

dCTP: Deoxycytidine triphosphate

ATP: Adenosine triphosphate

EDTA: Ethylenediaminetetracetic acid

SDS: Sodium dodecyl sulfate

Gly: Glycine

Ala: Alanine

Val: Valine

Leu: Leucine

Ile: Isoleucine

Ser: Serine

Thr: Threonine

Cys: Cysteine

Met: Methionine

Glu: Glutamic acid

Asp: Aspartic acid

Lys: Lysine

Arg: Arginine

His: Histidine

Phe: Phenylalanine

Tyr: Tyrosine

Trp: Tryptophan

Pro: Proline

Asn: Asparagine

Gln: Glutamine

pGlu: Pyroglutamic acid

The substituents, protective groups and reagents which are frequently used in the specification are represented by the following abbreviations.

Me: Methyl

Et: Ethyl

Bu: Butyl

Ph: Phenyl

TC: Thiazolidine-4(R)-carboxamide

Tos: p-Toluene sulfonyl

CHO: Formyl

Bzl: Benzyl

Cl₂Bzl: 2,6-Dichlorobenzyl

Bom: Benzyloxymethyl

Z: Benzyloxycarbonyl

Cl-Z: 2-Chlorobenzyloxycarbonyl

Br-Z: 2-Bromobenzyloxycarbonyl

Boc: t-Butoxycarbonyl

DNP: Dinitrophenyl

Trt: Trityl

Bum: t-Butoxymethyl

Fmoc: N-9-Fluorenylmethyloxycarbonyl

HOBt: 1-Hydroxybenzotriazole

HOOBt: 3,4-Dihydro-3-hydroxy-4-oxo-1,2,3-benzotriazine

HONB: 1-Hydroxy-5-norbornene-2,3-dicarboximide

DCC: N,N′-Dicyclohexylcarbodiimide

The SEQ ID NOS of the SEQUENCE LISTING of the present specification represent the sequences as indicated below.

[SEQ ID NO: 1]

This shows-the nucleotide sequence of the novel human cDNA fragment obtained in Example 1.

[SEQ ID NO: 2]

This shows the amino acid sequence of a precursor polypeptide.

[SEQ ID NO: 3]

This shows the nucleotide sequence of a DNA encoding a precursor polypeptide.

[SEQ ID NO: 4]

This shows the amino acid sequence of the polypeptide of the invention.

[SEQ ID NO: 5]

This shows the nucleotide sequence of a DNA encoding the polypeptide of the invention.

[SEQ ID NO: 6]

This shows the amino acid sequence of mature peptide 1.

[SEQ ID NO: 7]

This shows the nucleotide sequence of the DNA encoding mature peptide 1.

[SEQ ID NO: 8]

This shows the amino acid sequence of mature peptide 2.

[SEQ ID NO: 9]

This shows the nucleotide sequence of the DNA encoding mature peptide 2.

[SEQ ID NO: 10]

This shows the nucleotide sequence of a primer (sense strand) used in Example 1.

[SEQ ID NO: 11]

This shows the nucleotide sequence of a primer (antisense strand) used in Example 1.

[SEQ ID NO: 12]

This shows the nucleotide sequence of a primer (sense strand) used in Example 3.

[SEQ ID NO: 13]

This shows the nucleotide sequence of a primer (antisense strand) used in Example 3.

[SEQ ID NO: 14]

This shows the amino acid sequence of the polypeptide encoded by the expression vector constructed in Example 3.

[SEQ ID NO: 15]

This shows the nucleotide sequence coding for the polypeptide encoded by the expression vector constructed in Example 3.

[SEQ ID NO: 16]

This shows the nucleotide sequence of a primer (antisense strand) used in Example 6.

[SEQ ID NO: 17]

This shows the amino acid sequence of synthetic peptide CPP1-1 used in Example 8.

[SEQ ID NO: 18]

This shows the amino acid sequence of synthetic peptide CPP1-2 used in Example 8.

[SEQ ID NO: 19]

This shows the nucleotide sequence of a primer (antisense strand) used in Example 9.

[SEQ ID NO: 20]

This shows the nucleotide sequence of a primer (sense strand) used in Example 11.

[SEQ ID NO: 21]

This shows the nucleotide sequence of a primer (antisense strand) used in Example 11.

[SEQ ID NO: 22]

This shows the amino acid sequence of the mouse precursor polypeptide obtained in Example 11.

[SEQ ID NO: 23]

This shows the nucleotide sequence of the DNA encoding the mouse precursor polypeptide obtained in Example 11.

Transformant Escherichia coli XL10-Gold/pDRL90h obtained in Example 1 described later has been deposited with the Institute for Fermentation, Osaka (IFO) since Sep. 7, 2000 under the Accession No. IFO 16474 and has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) located at Central 6, 1-1 Higashi 1-chome, Tsukuba City, Ibaraki Pref. since Sep. 25, 2000 under the Accession No. FERM BP-7307.

### EXAMPLES

Hereinbelow, the present invention will be described more specifically with reference to the following Examples. However, the present invention is not limited to these Examples. Genetic operations using E. coli were carried out in accordance with the methods described in the book “Molecular Cloning”.

### Example 1

Cloning of cDNA Encoding Human DRL90

A sequence having both a signal sequence for secretion and basic amino acid pairs (such as Arg-Arg, Lys-Arg) in one same open reading frame (ORF) was searched for through human genetic information databases. As a result, a sequence from chromosome 17 was found in human gene sequences. In order to determine whether an mRNA comprising this gene sequence is actually transcribed or not and whether the predicted ORF is correct or not, a cDNA fragment was obtained as described below.

To 3 l each of human thymus-derived cDNA (Multiple Tissue cDNA Panel; Clontech K1421-1) solution, 10 pmol of oligo DNA: GGAACCAAGGCAGGATGAAGAC (SEQ ID NO: 10), 10 pmol of oligo DNA: GGTCCTGATCCCCCTTACACAG (SEQ ID NO: 11), 10 l of distilled water and 20 l of premix Taq (Ex Taq Version; Takara Shuzo) were added to give a total volume of 40l. In a thermal cycler (GeneAmp PCR System Model 9700; Perkin Elmer), the resultant mixture was reacted at 96 C for 1 min, followed by 35 cycles of reactions (94 C for 30 sec, 65 C for 1 min). As a result, an approx. 0.3 kb PCR fragment was amplified. Then, this fragment was purified by agarose gel electrophoresis, and cloned into pCR2.1-TOPO (Invitrogen) to determine the nucleotide sequence. The resultant plasmid was introduced into E. coli XL10-Gold competent cells (Stratagene). From colonies of ampicillin resistant transformant clones growing on ampicillin-containing LB agar medium, those clones retaining the plasmid having the 0.3 kb DNA fragment inserted thereinto were selected. Then, the plasmid DNA (pDRL90h) was prepared. In order to determine the nucleotide sequence of the inserted DNA, sequencing reaction was carried out using pDRL90h as a template DNA, two primer DNAs (PRM-007 and PRM-008; Toyobo) as sequencing primers and ABI PRISM™ BigDye Terminator Cycle Sequencing FS Ready Reaction Kit (Perkin Elmer) and analyzed with DNA Sequencer ABI PRISM™ 377 (Perkin Elmer).

As a result, it was revealed that a novel cDNA fragment consisting of the 301 bp nucleotide sequence as shown in SEQ ID NO: 1 having no homology to known nucleotide sequences was inserted into pDRL90h. This cDNA contained the nucleotide sequence of an open reading frame as shown in SEQ ID NO: 3 that encodes a novel protein (hereinafter, sometimes referred to as “DRL90h polypeptide” or “DRL90h”) consisting of the 90 amino acid residues as shown in SEQ ID NO: 2. From these results, it was confirmed that this gene is transcribed and translated. Furthermore, since a peptide-encoding open reading frame having no homology to known amino acid sequences was confirmed, it was clear that the above-described cDNA is a cDNA encoding a novel polypeptide comprising a signal sequence.

### Example 2

Analysis of Expression Sites in Human

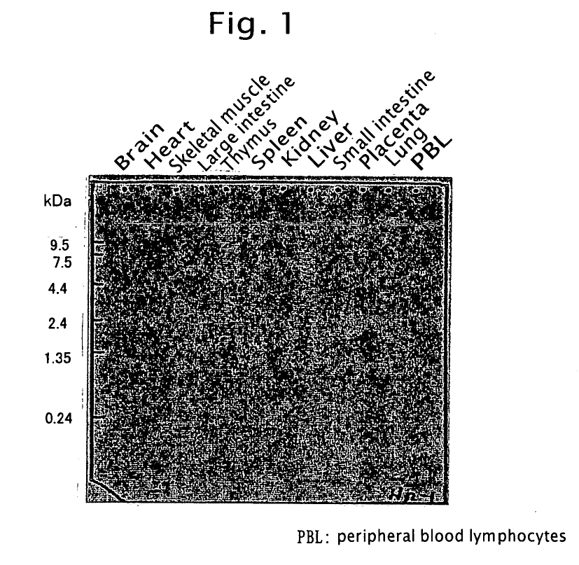

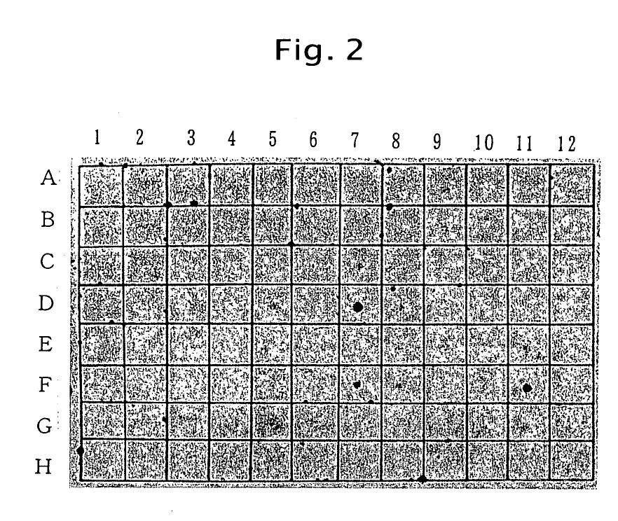

A DNA probe was prepared using 20 ng of the inserted DNA fragment described in Example 1 (0.3 kb EcoRI-EcoRI fragment) and 5 l of [-³²P] dCTP (Amersham: 6000 Ci/mmol) and following the protocol of Multiprime DNA Labeling System (Amersham: RPN. 1601Y). The resultant probe was hybridized to MTN Blot (Clontech #7780-1) and Human Multiple Tissue Expression Array (Clontech #7775-1). Hybridization and washing conditions were in accordance with the attached manuals. For detection, BAS-2000 (Fuji Film) was used. As a result, it was revealed that the mRNA of the clone was expressed in restricted tissues such as thymus (adult and fetus), spleen, liver, pancreas, lymph nodes and testis; it has become clear that the mRNA is expressed organ-specifically. The size of the mRNA was approx. 1.3 kb (FIGS. 1 and 2).

### Example 3

Construction of an Expression Vector for Human DRL90

A DNA fragment encoding human DRL90 was obtained by PCR as described below. A 50 l reaction mixture containing 20 pmol each of an oligo DNA represented by SEQ ID NO: 12 (5′-CTAGAATTCCACCATGAAGA CATTGCCTGTGCTCGTGCTG TCTCTTA-3′) as a sense strand primer and another oligo DNA represented by SEQ ID NO: 13 (5′-GATCCGTCGACCACAGGATG AATCCTGTTCCTGTCACAGT-3′) as an antisense strand primer, 5 l of 10× Advantage™ 2 PCR Buffer (Clontech), 1 l of 50× dNTP mix (Clontech), 1 l of 50× Advantage 2 polymerase Mix (Clontech) and 1 ng of the 0.3 kb fragment described in Example 1 as a template DNA was prepared. In a thermal cycler (GeneAmp PCR System Model 9700; Applied Biosystems), a PCR reaction was carried out. Reaction programs were as follows: 96 C for 1 min; 20 cycles of 96 C for 5 sec, 66 C for 5 sec and 72 C for 30 sec; and then 72 C for 1 min for elongation. After completion of the reaction, the reaction mixture was double-digested with restriction enzymes EcoRI and SalI (both Takara Shuzo). Then, unreacted primers and short DNA fragments generated by the restriction enzyme digestion were removed with QIAquick Gel PCR Purification Kit (Qiagen). The resultant fragment (0.28 kb) was sub-cloned into pCAN618.FLAG vector, which was then introduced into competent cells of an E. coli strain Epicurian Coli™ XL10-Gold™ (Stratagene). From colonies of ampicillin resistant transformant clones growing on ampicillin-containing LB agar medium, those clones retaining the plasmid having the foreign DNA fragment inserted thereinto were selected. Then, the plasmid DNA (pDRL90h-FLAG) was prepared.

Plasmid pDRL90h-FLAG comprises a DNA fragment of the 300 base pairs shown in SEQ ID NO: 15 encoding a human DRL90h-FLAG fusion protein in which a Val residue and a FLAG sequence Asp-Tyr-Lys-Asp-Asp-Asp-Asp-Lys follow the C-terminal of DRL90h polypeptide (as shown in SEQ ID NO: 14). FLAG sequence plays the role of an epitope (antibody recognition site) for detecting gene products of interest.

### Example 4

Expression in COS-7 Cells

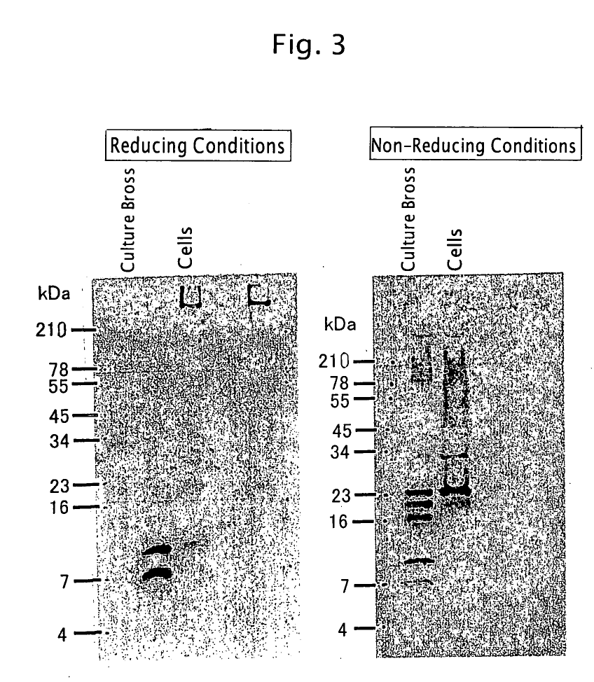

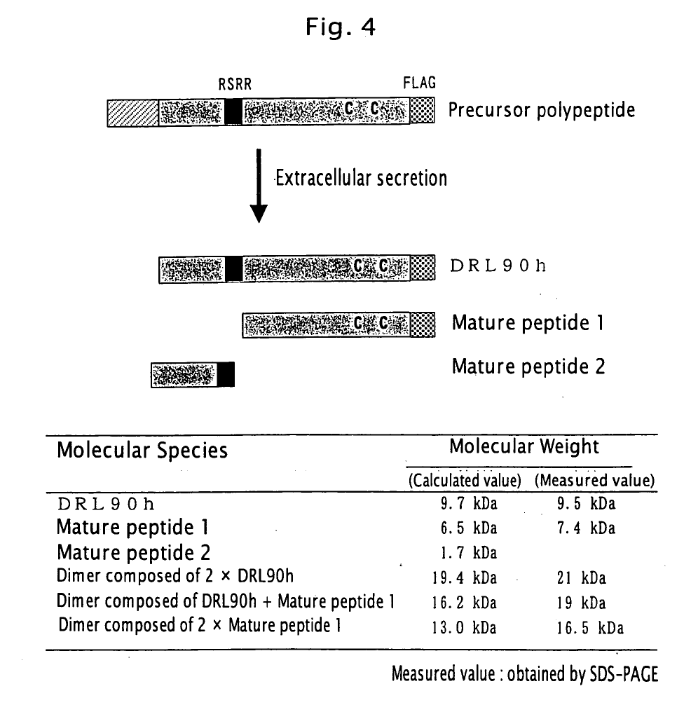

Expression experiments were conducted using COS-7 cells in order to examine whether a protein expressed by the expression vector is the human DRL90h-FLAG fusion protein or not; whether the expressed protein undergoes processing (restrictive degradation); and whether the expressed protein forms a multimer. The day before the transfection of the expression vector, COS-7 cells were seeded on 6-well plates at 2×10⁵ cells/well. Then, the cells were incubated in 10% FBS (JRH)-containing DMEM medium (Gibco BRL) for 24 hr in a CO₂ incubator. Transfection was carried out using 1 g/well of the pDRL90h-FLAG DNA constructed in Example 3 and Effectene (Qiagen). Twenty-four hours after the transfection, the medium was replaced with serum-free DMEM medium supplemented with 1 ml of 0.1 mM pABSF (Wako Purechemical Industries) and 0.05% CHAPS (Dojin Chemical). Then, the cells were cultured further for 48 hr. The culture supernatant was transferred into Eppendorf sample tubes and centrifuged. After removal of floating cells, the supernatant was concentrated to about 1/10 in volume with Centricon YM-3 ultrafiltration membrane (Millipore). An equal volume of Tricine SDS-PAGE sample buffer (TEFCO) was added to the resultant concentrated culture supernatant. With respect to the cells, they were washed in 1 ml of PBS (Gibco BRL) twice and then 500 l of Tricine SDS-PAGE sample buffer was added thereto. Since DRL90 polypeptide has two cysteine residues, electrophoresis should be carried out both under non-reducing conditions and under reducing conditions. Ten percent (w/v) DTT was added only to electrophoresis samples to be used under reducing conditions. After thermal treatment at 95 C for 5 min, these samples were electrophoresed on 16% Peptide-PAGE mini (TEFCO). The electrophoresed polypeptide was transferred from the gel onto a nitrocellulose filter. After blocking the nitrocellulose filter with PBS containing 0.1% Tween 20 and 50% Block Ace (Snow Brand Milk Products), human DRL90-FLAG fusion protein was detected with antibodies. As a primary antibody, anti-FLAG M2 mouse IgG monoclonal antibody (1/2000 dilution; Sigma) was used. As a secondary antibody, HRP-labeled anti-mouse IgG sheep antibody (1/2000 dilution; Amersham Pharmacia Biotech) was used. For luminescence, ECL Western Blotting Kit (Amersham Pharmacia Biotech) was used. Chemiluminescence was detected with Hyperfilm ECL (Amersham Pharmacia Biotech). The results revealed that human DRL90-FLAG fusion protein was secreted into cell culture supernatant for the most part; that the molecular weights of the protein were approx. 7.1 kDa and 9.2 kDa under reducing conditions and approx. 7.4 kDa, 9.5 kDa, 16.5 kDa, 19 kDa and 21 kDa under non-reducing conditions (FIG. 3). The fusion protein in cell fractions was detected as a polypeptide of approx. 9.2 kDa under reducing conditions and as a polypeptide of approx. 21 kDa under non-reducing conditions. From the above, it has been found that human DRL90-FLAG fusion protein exists not only in the form of a monomer but it forms dimers through the cysteine residues in the polypeptide. It was also suggested that, in the process of extracellular secretion of the protein, a polypeptide of approx. 9 kDa in a monomer form is divided into a polypeptide of approx. 7 kDa and a polypeptide of approx. 2 kDa through restrictive degradation (processing) by proteases, and that the protein exists in the forms of three types of molecules as dimers. Also, it is considered that the restrictive degradation occurs at the cleavage sequence (Arg-Ser-Arg-Arg) of furin, a protein processing enzyme (FIG. 4).

### Example 5

Expression in CHO Cells

Chinese hamster ovary cells (CHO-K1) were cultured in Ham's F-12 medium containing 10% fetal bovine serum (FBS). The expression plasmid pDRL90h-FLAG constructed in Example 3 (1 l) was introduced into the cells using Effectene (Qiagen). Subsequently, the cells were cultured in a culture broth containing 500 g/ml of G418. Surviving cells (transformant cells into which the plasmid had been introduced) were selected. The resultant G418-resistant CHO cells were cultured in a culture broth or Ham F-12 medium that does not contain fetal bovine serum tentatively to thereby obtain a culture supernatant where gene products (human type polypeptides) have been produced.

### Example 6

Construction of an Expression Vector for Human DRL90 (Part 2)

A DNA fragment encoding human DRL90 was obtained by PCR as described below. A 50 l reaction mixture containing 20 pmol each of an oligo DNA represented by SEQ ID NO: 12 as a sense strand primer and another oligo DNA represented by SEQ ID NO: 16 as an antisense strand primer, 5 l of 10× Advantage™ 2 PCR Buffer (Clontech), 1 l of 50× dNTP mix (Clontech), 1 l of 50× Advantage 2 Polymerase Mix (Clontech) and 5 l of human pancreas cDNA solution as a template DNA was prepared. In a thermal cycler (GeneAmp™ PCR System Model 9700; Applied Biosystems), a PCR reaction was carried out. Reaction programs were as follows: 96 C for 1 min; 30 cycles of 96 C for 5 sec, 66 C for 5 sec and 72 C for 30 sec; and then 72 C for 1 min for elongation. After completion of the reaction, the reaction mixture was double-digested with restriction enzymes EcoRI and SalI (both Takara Shuzo). Then, unreacted primers and short DNA fragments generated by the restriction enzyme digestion were removed with QlAquick Gel PCR Purification Kit (Qiagen). The resultant fragment was sub-cloned into an animal cell expression vector pCAN618 (WO 00/14226), which was then introduced into competent cells of an E. coli strain Epicurian Coli™ XL10-Gold™ (Stratagene). From colonies of ampicillin resistant transformant clones growing on ampicillin-containing LB agar medium, those clones retaining the plasmid having the foreign DNA fragment inserted thereinto were selected. Then, the plasmid DNA (pCAN618/hCPP1) was prepared.

As a result of analysis of its nucleotide sequence, it was found that pCAN618/hCPP1 contained a DNA fragment of the 270 base pairs shown in SEQ ID NO: 3 encoding a precursor polypeptide consisting of the 90 amino acids shown in SEQ ID NO: 2.

### Example7

Establishment of Human DRL90-Expressing CHO-KI Cell Strain

CHO-K1 cells (3.3×10⁴ cells/dish) were cultured in F-12 medium (Gibco BRL) containing 10% fetal bovine serum (FBS) for 24 hr in petri dishes 10 cm in diameter. The expression plasmid pCAN618/hCPP1 (1.5 g/dish) was introduced into the cells by the phosphate calcium method using CellPhect Transfection Kit (Pharmacia). Twelve hours after the introduction, cells were washed with FBS-free F-12 medium twice, and then glycerol shock was given to the cells for 3 min using 3 ml of isotonic HEPES solution (pH 7.5) containing 15% glycerol. After washing with FBS-free F-12 medium twice, the cells were cultured in FBS-containing F-12 medium for another 12 hr. Then, the medium was replaced with F-12 selection medium containing 500 mg/L Geneticine and 10% FBS (hereinafter, called the “selection medium”). Ten days thereafter, colonies formed in the petri dishes were transferred individually into 24-well plates and cultured in the selection medium for 3 days. Cells grown in the selection medium were transferred to 6-well plates and cultured further in the selection medium for 4 days. Then, the medium was replaced with 1 ml of Opti-MEM (Gibco BRL) containing 0.02% CHAPS and 0.1 mM pABSF (Wako Pure Chemical). The cells were cultured for another 48 hr and then the culture supernatant was recovered. After concentration of the culture supernatant with Centricon YM-3 ultrafiltration membrane (Millipore), an equal volume of Tris-Tricine SDS sample buffer was mixed with the supernatant. This sample was treated at 95 C for 5 min and then electrophoresed on 16% Peptide-PAGE mini (TEFCO). The electrophoresed polypeptides were transferred from the gel onto a nylon membrane. After blocking with Block Ace (Snow Brand Milk Products) for 1 hr, the nylon membrane was reacted with the anti-human DRL90 antibody prepared in Example 8 (TAL-100108-S; 1/2006 dilution) for 1 hr in 0.05% Tween 20-containing PBS (PBS-T). After washing with PBS-T 5 times, the nylon membrane was reacted with an HRP-labeled anti-rabbit IgG sheep antibody (1/2000 dilution; Amersham Pharmacia Biotech) for 1 hr in PBS-T. After washing the nylon membrane with PBS-T 5 times, chemiluminescence was detected with ECL color development kit (Amersham Pharmacia Biotech) and Hyperfilm ECL (Amersham Pharmacia Biotech). As a result, approx. 6 kDa and 8 kDa recombinant products were detected in the cell culture supernatant. From these results, it was confirmed that a human DRL90-expressing CHO-K1 cell strain had been successfully established.

### Example 8

Preparation of Anti-Human DRL90 Antibody

In order to detect anti-human DRL90, anti-sera were obtained by immunizing rabbits with two synthetic peptides. Briefly, cysteine-introduced two synthetic polypeptides, i.e. CPP1-1 (N-QDRPSKPGFPC; SEQ ID NO: 17) and CPP1-2 (N-CSPILTEKQAK; SEQ ID NO: 18), were prepared and conjugated to a carrier protein (keyhole limpet hemocyanin; KLH) by the maleimide method. With these conjugated antigens, rabbits were immunized to thereby obtain anti-sera TAL-100108-S and TAL-100107-S, respectively. Subsequently, the human DRL90h-FLAG fusion protein expressed in COS-7 cells in Example 4 was detected by Western blotting using the resultant anti-sera. As a result, anti-serum TAL-100107-S obtained by using synthetic peptide CPP1-2 (which corresponds to a partial amino acid sequence of human DRL90 located on the N-terminal side of the furin cleavage site within the protein) only detected a 9.2 kDa molecule that is not cleaved at the furin cleavage site. On the other hand, anti-serum TAL-100108-S obtained by using synthetic peptide CPP1-1 (which corresponds to a partial amino acid sequence of human DRL90 located on the C-terminal side of the furin cleavage site within the protein) detected two molecular species, i.e. a 7.1 kDa molecule cleaved at the furin cleavage site and a 9.2 kDa molecule not cleaved. These molecular species could also be detected with anti-FLAG antibody that recognizes the FLAG tag present at the C-terminal of the human DRL90h-FLAG fusion protein. These results revealed that it is possible to detect anti-human DRL90 with the resultant rabbit anti-sera.

### Example9

Identification of DRL90-Expressing Cell Strains

As described in Example 2, the mRNA of human DRL90 is expressed in restricted tissues such as thymus (adult and fetus), spleen, liver, pancreas, lymph nodes and testis. Then, expression of DRL90 in blood cell-type cell strains was analyzed first by RT-PCR. This analysis was carried out using 12 leukemia cell strains: K-562, HL-60, THP-1, U937 DF-4, MEG-01, EoL-1, KU812, BALL-1, U266BL, CRRF-HSB-2, Molt-4 and H9. First, total RNA was prepared from each cell strain using TRIzol reagent (Gibco BRL). Poly (A)⁺ RNA was prepared from the total RNA using mRNA Purification Kit (Amersham Pharmacia Biotech). After treatment with DNaseI (Gibco BRL), the RNA was converted into cDNA using S_{UPER}S_{CRIPT}™ First-Strand Synthesis System for RT-PCR (Gibco BRL) and used as a template in the subsequent RT-PCR described below. Briefly, a 50 l reaction mixture containing 20 pmol each of an oligo DNA represented by SEQ ID NO: 10 as a sense strand primer and another oligo DNA represented by SEQ ID NO: 19 as an antisense strand primer, 5 l of 10× Advantage^{TM} 2 PCR Buffer (Clontech), 1 l of 50× dNTP (Clontech), 1 l of 50× Advantage 2 Polymerase Mix (Clontech) and 5 l of template DNA solution was prepared. In a thermal cycler (GeneAmp™ PCR System Model 9700; PE Biosystems), a PCR reaction was carried out. Reaction programs were as follows: 96 C for 1 min; 30 cycles of 96 C for 5 sec and 68 C for 30 sec; and then 68 C for 1 min for elongation. As a result, the expression of DRL90 was confirmed in all the cell strains analyzed. Among all, considerably high expression was observed in the bone marrow cell strains of U937 DF-4, MEG-01 and EoL-1; and all of the analyzed lymphocyte cell strains of BALL-1, U266BL, CCRF-HSB-2, Molt-4 and H9.

### Example 10

Identification of DRL90-Expressing Cells in Testis and Epididymis

As described in Example 2, the results of Northern analysis revealed that DRL90 is expressed highly in testis and epididymis. Then, in order to elucidate the function of DRL90, the expression of DRL90 in normal mouse testis and epididymis was examined by immunological staining with the rabbit anti-sera prepared in Example 8.

Eleven week-old normal male ICR mice were perfusion fixed with 4% paraformaldehyde under ether anesthetization. After removal of the testis and epididymis, both tissues were fixed with 4% paraformaldehyde overnight at 4 C. After washing these tissues with water, paraffin embedded blocks were prepared according to routine procedures. (“Methods for Studying Histology”, Yutaka Sano, Nanzando, 1985). The thus prepared blocks were cut into sections 4 μm in thickness with a rotary microtome. These sections were extended in hot bath, mounted on slide glass and dried sufficiently in a dryer at 37 C. After deparaffinization, the thus prepared sections were stained with hematoxylin-eosin and enclosed according to routine procedures. (“Methods for Studying Histology”, Yutaka Sano, Nanzando, 1985). Further, the thus prepared sections were subjected to immunological staining with the rabbit anti-serum against DRL90 (TAL-100108-S). After deparaffinization of the slide glass on which the section was mounted, immunological staining was carried out with VECTASTAIN ABC Kit (Peroxidase method; Vector). As a substrate for color development, DAB was used. Specific experimental procedures were in accordance with the manual attached to the kit.

In the testis, expression of DRL90 was observed in sperm cells and sperm. In the epididymis, expression of DRL90 was localized to epithelial cells of the efferent ductules of testis and laeve epithelial cells of the head of the duct of epididymis. Because expression of DRL90 was observed in highly differentiated cells among male germ cells in the testis and because no expression was observed in Sertoli cells and Leydig cells that constitute the testis parenchyma, it was conjectured that DRL90 gene is involved in the differentiation of sperm. It was also conjectured that this gene is involved in the movement of sperm to the epididymis and the conferring of reproductive functions because expression of DRL90 was observed in the efferent ductules of the testis which lead sperm from the testis to the epididymis and in the head of the duct of epididymis.

### Example 11

Creation of Transgenic Mouse

A DNA fragment encoding mouse DRL90 was obtained by PCR as described below.

Briefly, a 50 l reaction mixture containing 20 pmol each of an oligo DNA represented by SEQ ID NO: 20 as a sense strand primer and another oligo DNA represented by SEQ ID NO: 21 as an antisense strand primer, 5 l of 10× Advantage™ 2 PCR Buffer (Clontech), 1 l of 50× dNTP mix (Clontech), 1 l of 50× Advantage 2 Polymerase Mix (Clontech) and 5 l of mouse testis cDNA solution as a template DNA was prepared. In a thermal cycler (GeneAmp™ PCR System Model 9700; PE Biosystems), a PCR reaction was carried out. Reaction programs were as follows: 96 C for 1 min; 30 cycles of 96 C for 5 sec, 60 C for 5 sec and 72 C for 30 sec; and then 72 C for 2 min for elongation. After completion of the reaction, the reaction mixture was double-digested with restriction enzymes EcoRI and SalI (both Takara Shuzo). Then, unreacted primers and short DNA fragments generated by the restriction enzyme digestion were removed with QIAquick Gel PCR Purification Kit (Qiagen). The resultant fragment was sub-cloned into an animal cell expression vector pCAN618 (WO 00/14226), which was then introduced into competent cells of an E. coli strain Epicurian Coli™ XL10-Gold™ (Stratagene). From colonies of ampicillin resistant transformant clones growing on ampicillin-containing LB agar medium, those clones retaining the plasmid having the foreign DNA fragment inserted thereinto were selected. Then, the plasmid DNA (pCAN616/mCPP1) was prepared.

As a result of analysis of its nucleotide sequence, it was found that pCAN616/mCPP1 contained a DNA fragment of the 270 base pairs shown in SEQ ID NO: 23 encoding mouse DRL90 consisting of the 90 amino acids shown in SEQ ID NO: 22.

A mouse DRL90 gene fragment comprising a promoter sequence and a terminator sequence, which is a gene fragment for injection to be used in the creation of a transgenic mouse, was prepared as described below.

Briefly, pCAN616/mCPP1 was double-digested with BamHI and HindIII (both Takara Shuzo), extracted with 50:50 phenol/chloroform solution twice for removal of proteins, and then subjected to ethanol precipitation to thereby obtain a DNA fragment. Subsequently, the DAN fragment was electrophoresed on 1% SeaKem GTG agarose gel (Takara Shuzo) with TAE electrophoresis buffer to thereby separate an approx. 2.3 kb DNA fragment. This fragment was cut out from the gel and recovered with QIAquick Gel Extraction Kit (Qiagen). The resultant DNA solution was treated with 50:50 phenol/chloroform, washed with 70% ethanol three times and subjected to ethanol precipitation to thereby recover the DNA fragment of interest. After air drying, the DNA fragment was re-suspended in 1 mM Tris-0.1 mM EDTA (pH 8.0) solution to thereby obtain a mouse DRL90 gene fragment to be used in the creation of a transgenic mouse. This suspension, after dilution to give a concentration of 5 ng/1, is microinjected into mouse fertilized egg cells, from which transgenic mice are created according to conventional methods.

### Example 12

Physicochemical Analysis of Human DRL90 Gene Product Produced by Secretion

The COS-7 cell-derived human DRL90h-FLAG fusion protein described in Example 4 was purified, and molecular species thereof were analyzed physicochemically. For purification, 80 ml of the culture supernatant was dialyzed against TBS (50 mM Tris-HCl, 150 mM NaCl, pH 7.4) containing 0.1 mM pABSF and 0.05% CHAPS, and then applied to anti-FLAG M2 antibody column (Sigma). The antibody column was washed with 40 ml of TBS containing 0.1 mM pABSF and 0.05% CHAPS, and then eluted with 12 ml of glycine hydrochloride (pH 3.0). The eluate was desalted and concentrated with C18 Bioselect SPE cartridge (VYDAC), followed by washing with 30% acetonitrile, 0.1% TFA and elution with 50% acetonitrile, 0.1% TFA. Finally, a purified sample was obtained as a single peak fraction by HPLC (ODS-80TS column; Solution A: 30% acetonitrile, 0.1% TFA; Solution B: 50% acetonitrile, 0.1% TFA; Solution B was increased from 0% to 100% in 60 min).

The results of mass spectrometric analysis by LC/MS method revealed that the measured molecular weight was 7552.0, which was almost equal to the theoretical molecular weight (7551.4) of a molecular species where the N-terminal glutamine is converted to pyroglutaminate and a pair of intramolecular disulfide bonds are formed. Therefore, it was found that the major component of the human DRL90 gene product is a molecular species wherein the N-terminal of the 53 amino acid residues shown in SEQ ID NO: 6 is pyroglutaminate and a pair of intramolecular disulfide bonds are formed.

### INDUSTRIAL APPLICABILITY

The polypeptide and the DNA encoding the same of the invention can be used for the diagnosis, treatment and/or prevention of various diseases, e.g. cancers, immunological diseases, infections, gastrointestinal diseases, circulatory organ diseases and endocrine diseases. The polypeptide of the invention is also useful as a reagent for screening for those compounds or salts thereof which promote or inhibit the activity of the polypeptide of the invention. Further, since antibodies to the polypeptide of the invention can recognize the polypeptide of the invention specifically, they can be used in the detection, quantitative determination or neutralization of the polypeptide of the invention in sample solutions.

1. A polypeptide comprising an amino acid sequence identical or substantially identical with the amino acid sequence as shown in SEQ ID NO: 4, or an amide, ester or salt thereof.

2. The polypeptide according to claim 1 comprising the amino acid sequence as shown in SEQ ID NO: 4, SEQ ID NO: 2 or SEQ ID NO: 14, or an amide, ester or salt thereof.

3. A peptide comprising an amino acid sequence identical or substantially identical with the amino acid sequence as shown in SEQ ID NO: 6 or SEQ ID NO: 8, or an amide, ester or salt thereof.

4. The peptide according to claim 3 comprising the amino acid sequence as shown in SEQ ID NO: 6 or SEQ ID NO: 8, or an amide, ester or salt thereof.

5. A partial peptide of the polypeptide according to claim 1 or the polypeptide according to claim 3, or an amide, ester or salt of said partial peptide.

6. A polynucleotide comprising a polynucleotide having a nucleotide sequence encoding the polypeptide according to claim 1.

7. The polynucleotide according to claim 6 comprising a polynucleotide having the nucleotide sequence as shown in SEQ ID NO: 1, SEQ ID NO: 3, SEQ ID NO: 5 or SEQ ID No: 15.

8. The polynucleotide according to claim 6 or 7, wherein the polynucleotide is DNA.

9. A polynucleotide comprising a polynucleotide having a nucleotide sequence encoding the peptide according to claim 3.

10. The polynucleotide according to claim 9 comprising a nucleotide having the nucleotide sequence as shown in SEQ ID NO: 7 or SEQ ID NO: 9.

11. The polynucleotide according to claim 9 or 10, wherein the polynucleotide is DNA.

12. A recombinant vector comprising the polynucleotide according to claim 6 or 9.

13. A transformant transformed with the recombinant vector according to claim 12.

14. A method for producing the polypeptide according to claim 1 or an amide, ester or salt thereof or the peptide according to claim 3 or an amide, ester or salt thereof, comprising culturing the transformant and allowing the polypeptide according to claim 1 or the peptide according to claim 3 to be produced and accumulated.

15. An antibody to the polypeptide according to claim 1 or an amide, ester or salt thereof, the peptide according to claim 3 or an amide, ester or salt thereof, or the partial peptide according to claim 5 or an amide, ester or salt thereof.

16. A method for screening for compounds, or salts thereof, that promote or inhibit the activity of the polypeptide according to claim 1 or an amide, ester or salt thereof, the peptide according to claim 3 or an amide, ester or salt thereof, or the partial peptide according to claim 5 or an amide, ester or salt thereof, wherein the method is characterized by using the polypeptide according to claim 1 or an amide, ester or salt thereof, the peptide according to claim 3 or an amide, ester or salt thereof, or the partial peptide according to claim 5 or an amide, ester or salt thereof.

17. A kit for screening for compounds, or salts thereof, that promote or inhibit the activity of the polypeptide according to claim 1 or an amide, ester or salt thereof, the peptide according to claim 3 or an amide, ester or salt thereof, or the partial peptide according to claim 5 or an amide, ester or salt thereof, which comprises the polypeptide according to claim 1 or an amide, ester or salt thereof, the peptide according to claim 3 or an amide, ester or salt thereof, or the partial peptide according to claim 5 or an amide, ester or salt thereof.

18. A compound, or a salt thereof, that promotes or inhibits the activity of the polypeptide according to claim 1 or an amide, ester or salt thereof, the peptide according to claim 3 or an amide, ester or salt thereof, or the partial peptide according to claim 5 or an amide, ester or salt thereof, which is obtainable by using the screening method according to claim 16 or the screening kit according to claim 17.

19. A medicine comprising the compound according to claim 18 or a salt thereof.

20. A prophylactic and/or therapeutic agent for cancers, immunological diseases, infections, gastrointestinal diseases, circulatory organ diseases or endocrine diseases, comprising a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

21. A prophylactic and/or therapeutic agent for sterility, comprising a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

22. An agent for regulating the differentiation and/or function of germ cells, comprising a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

23. An agent for regulating the differentiation and/or proliferation of blood cells, comprising a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

24. A medicine comprising the polypeptide according to claim 1 or an amide, ester or salt thereof, or the peptide according to claim 3 or an amide, ester or salt thereof.

25. The medicine according to claim 24, wherein said medicine is a prophylactic and/or therapeutic agent for cancers, immunological diseases, infections, gastrointestinal diseases, circulatory organ diseases or endocrine diseases.

26. The medicine according to claim 24, wherein said medicine is a prophylactic and/or therapeutic agent for sterility.

27. The medicine according to claim 24, wherein said medicine is an agent for regulating the differentiation and/or function of germ cells.

28. The medicine according to claim 24, wherein said medicine is an agent for regulating the differentiation and/or proliferation of blood cells.

29. A medicine comprising the polynucleotide according to claim 6 or 9.

30. The medicine according to claim 29, wherein said medicine is a prophylactic and/or therapeutic agent for cancers, immunological diseases, infections, gastrointestinal diseases, circulatory organ diseases or endocrine diseases.

31. The medicine according to claim 29, wherein said medicine is a prophylactic and/or therapeutic agent for sterility.

32. The medicine according to claim 29, wherein said medicine is an agent for regulating the differentiation and/or function of germ cells.

33. The medicine according to claim 29, wherein said medicine is an agent for regulating the differentiation and/or proliferation of blood cells.

34. A diagnostic agent comprising the antibody according to claim 15.

35. A medicine comprising the antibody according to claim 15.

36. An antisense DNA having a nucleotide sequence, or a part thereof, complementary or substantially complementary to the DNA encoding the polypeptide according to claim 1 or the peptide according to claim 3, said antisense DNA having an effect capable of inhibiting the expression of said DNA.

37. A medicine comprising the antisense DNA according to claim 36.

38. A dimer of a polypeptide comprising an amino acid sequence identical or substantially identical with the amino acid sequence as shown in SEQ ID NO: 4; or an amide, ester or salt of said dimer.

39. A dimer composed of two polypeptides comprising the amino acid sequence as shown in SEQ ID NO: 4, wherein the respective C-terminal cysteine residues of the two polypeptides are linked to each other; or an amide, ester or salt of said dimer.

40. A dimer of a peptide comprising an amino acid sequence identical or substantially identical with the amino acid sequence as shown in SEQ ID NO: 6; or an amide, ester or salt of said dimer.

41. A dimer composed of two peptides comprising the amino acid sequence as shown in SEQ ID NO: 6, wherein the respective C-terminal cysteine residues of the two peptides are linked to each other; or an amide, ester or salt of said dimer.

42. A dimer composed of a polypeptide comprising an amino acid sequence identical or substantially identical with the amino acid sequence as shown in SEQ ID NO: 4 and a peptide comprising an amino acid sequence identical or substantially identical with the amino acid sequence as shown in SEQ ID NO: 6; or an amide, ester or salt of said dimer.

43. A dimer composed of a polypeptide comprising the amino acid sequence as shown in SEQ ID NO: 4 and a peptide comprising the amino acid sequence as shown in SEQ ID NO: 6, wherein the respective C-terminal cysteine residues of the polypeptide and the peptide are linked to each other; or an amide, ester or salt of said dimer.

44. A method of preventing and/or treating cancers, immunological diseases, infections, gastrointestinal diseases, circulatory organ diseases or endocrine diseases, which is characterized by administering to a mammal an effective amount of the polypeptide according to claim 1 or an amide, ester or salt thereof or the peptide according to claim 3 or an amide, ester or salt thereof.

45. A method of preventing and/or treating sterility, which is characterized by administering to a mammal an effective amount of the polypeptide according to claim 1 or an amide, ester or salt thereof or the peptide according to claim 3 or an amide, ester of salt thereof.

46. A method of regulating the differentiation and/or function of germ cells, which is characterized by administering to a mammal an effective amount of the polypeptide according to claim 1 or an amide, ester or salt thereof or the peptide according to claim 3 or an amide, ester or salt thereof.

47. A method of regulating the differentiation and/or proliferation of blood cells, which is characterized by administering to a mammal an effective amount of the polypeptide according to claim 1 or an amide, ester or salt thereof or the peptide according to claim 3 or an amide, ester or salt thereof.

48. A method of preventing and/or treating cancers, immunological diseases, infections, gastrointestinal diseases, circulatory organ diseases or endocrine diseases, which is characterized by administering to a mammal an effective amount of the polynucleotide according to claim 6 or 9.

49. A method of preventing and/or treating sterility, which is characterized by administering to a mammal an effective amount of the polynucleotide according to claim 6 or 9.

50. A method of regulating the differentiation and/or function of germ cells, which is characterized by administering to a mammal an effective amount of the polynucleotide according to claim 6 or 9.

51. A method of regulating the differentiation and/or proliferation of blood cells of germ cells, which is characterized by administering to a mammal an effective amount of the polynucleotide according to claim 6 or 9.

52. A method of preventing and/or treating cancers, immunological diseases, infections, gastrointestinal diseases, circulatory organ diseases or endocrine diseases, which is characterized by administering to a mammal an effective amount of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

53. A method of preventing and/or treating sterility, which is characterized by administering to a mammal an effective amount of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

54. A method of regulating the differentiation and/or function of germ cells, which is characterized by administering to a mammal an effective amount of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

55. A method of regulating the differentiation and/or proliferation of blood cells, which is characterized by administering to a mammal an effective amount of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17.

56. Use of the polypeptide according to claim 1 or an amide, ester or salt thereof or the peptide according to claim 3 or an amide, ester or salt thereof for producing a prophylactic and/or therapeutic agent for cancers, immunological diseases, infections, gastrointestinal diseases, circulatory organ diseases or endocrine diseases.

57. Use of the polynucleotide according to claim 6 or 9 for producing a prophylactic and/or therapeutic agent for cancers, immunological diseases, infections, gastrointestinal diseases, circulatory organ diseases or endocrine diseases.

58. Use of the polynucleotide according to claim 6 or 9 for producing a prophylactic and/or therapeutic agent for sterility.

59. Use of the polynucleotide according to claim 6 or 9 for producing an agent for regulating the differentiation and/or function of germ cells.

60. Use of the polynucleotide according to claim 6 or 9 for producing an agent for regulating the differentiation and/or proliferation of blood cells.

61. Use of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17, for producing a prophylactic and/or therapeutic agent for cancers, immunological diseases, infections, gastrointestinal diseases, circulatory organ diseases or endocrine diseases.

62. Use of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17, for producing a prophylactic and/or therapeutic agent for sterility.

63. Use of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17, for producing an agent for regulating the differentiation and/or function of germ cells.

64. Use of a compound, or a salt thereof, that promotes the activity of the polypeptide according to claim 1 or an amide, ester or salt thereof, the peptide according to claim 3 or an amide, ester or salt thereof, or the partial peptide according to claim 5 or an amide, ester or salt thereof, said compound or salt thereof being obtainable by using the screening method according to claim 16 or the screening kit according to claim 17, for producing an agent for regulating the differentiation and proliferation of blood cells.

65. A non-human transgenic animal harboring the DNA according to claim 7 or 9 which is a foreign DNA or a mutant DNA thereof.

66. The animal according to claim 65, wherein the non-human animal is a rodent.

67. The animal according to claim 66, wherein the rodent is mouse or rat.

68. A recombinant vector comprising the DNA according to claim 7 or 9 which is a foreign DNA or a mutant DNA thereof, wherein the vector is capable of expression in a non-human animal.
